(19) United States
(12) Patent Application Publication
(10) Pub. No.: US 2019/0321050 A1
(43) Pub. Date: Oct. 24, 2019

(54) METHODS AND DEVICES FOR THE TREATMENT OF PULMONARY DISORDERS

(71) Applicant: EOLO MEDICAL INC., New York, NY (US)

(72) Inventors: Mark GELFAND, New York, NY (US); Anthony WONG, Franklin, MA (US); Robert F. RIOUX, Ashland, MA (US); Zoar ENGELMAN, New York, NY (US)

(73) Assignee: Eolo Medical Inc., New York, NY (US)

(21) Appl. No.: 16/317,419

(22) PCT Filed: Jul. 14, 2017
(86) PCT No.: PCT/US2017/042048

§ 371 Date: Jan. 11, 2019

Related U.S. Application Data
(60) Provisional application No. 62/362,330, filed on Jul. 14, 2016.

(30) Foreign Application Priority Data
Nov. 4, 2016 (CN) 201610962125.1

(51) Int. Cl. A61B 17/12; A61B 17/128; A61B 1/267; A61F 2/04

(52) U.S. Cl. CPC A61B 17/12104; A61B 17/1285; A61B 1/2676; A61B 2017/0034; A61B 17/12131; A61F 2/04; A61B 17/12031

(57) ABSTRACT

A medical device assembly including: an lung reduction device including a vertex, a first arm having an end connected to the vertex, and a second arm having an end connected to vertex, wherein the first and second arms extend into a respective one of airway branches in the lung and the vertex seats upstream of a bifurcation of the airway branches, wherein the first and second arms apply a bias force to the airway branches and thereby reduce a section of the lung near the airway branches; a bronchoscope including a channel housing the lung reduction device and having an opening to the channel through which the lung reduction device is deployed, and a pusher device associated with the bronchoscope and adapted to push the lung reduction device from the working channel to advance the first and second arms into the airway branches.

### FIELD OF THE INVENTION

The field of the invention is lung reduction devices used to treat chronic obstructive pulmonary disease (COPD). In particular, the invention relates to lung reduction devices configured to be delivered through the airway to the lung with minimally invasive techniques.

### BACKGROUND OF THE INVENTION

COPD is a lung disease that makes it hard to breathe. COPD can cause coughing that produces large amounts of phlegm or mucus, wheezing, shortness of breath, chest tightness, and other symptoms. Cigarette smoking is the leading cause of COPD, but long-term exposure to other lung irritants, such as air pollution, chemical fumes, or dust, may contribute to COPD. COPD is a progressive disease which gets worse over time, such as over the course of several years.

To understand COPD, it helps to understand how the lungs work. Air drawn in through the nose or mouth when drawing breath goes down the windpipe into tubes in the lungs called bronchi or airways. Within the lungs, the bronchi branch out into thousands of smaller, thinner tubes called bronchioles. These tubes end in bunches of tiny round air sacs called alveoli. Small blood vessels called capillaries run through the walls of the air sacs. When air reaches the air sacs, oxygen passes through the air sac walls into the blood in the capillaries. At the same time, carbon dioxide (a waste gas) moves from the capillaries into the air sacs. This process is called gas exchange. Typically, the airways and air sacs are elastic and may stretch to accommodate air intake. When a breath is drawn in, each air sac fills up with air like a small balloon. When a breath is expelled, the air sacs deflate and the air goes out. The expansion and contraction of the air sac are critical to the gas exchange. Air sacs that are free to expand exchange more gas than air sacs that are constricted or prevented from fully expanding.

In those with COPD, less air flows in and out of the airways because of one or more of the following: the airways and air sacs lose their elastic quality; the walls between many of the air sacs are destroyed; the walls of the airways become thick and inflamed, and the airways make more mucus than usual, which can result in mucus buildup and airway blockage.

In typical cases of COPD, the disease does not equally affect all air sacs or alveoli in a lung. A lung may have regions in which the air sacs are damaged and unsuited for gas exchange. In severe cases, these regions may be large, such as 20 to 30 percent or more of the lung volume. Thus, large regions of the lung may be damaged and unable to effectively perform the gas exchange. Alternatively, the damaged regions may be small islands of air sacs disbursed throughout the lung.

The effects of COPD are typically most debilitating when a patient exercises or engages in other physical excretion that would cause a healthy patient to breath heavily. A patient with COPD may not be able to breathe heavily because the diseased portions of the lung trap air that then results in the inability to exhale, or breathe out. This, in turn, prevents the subsequent expansion of healthy lung portions to their optimal size. During exercise or other physical exertion, the lung(s) of a patient affected by COPD may operate in dynamic hyperinflation of the lung(s), which impairs respiratory mechanics, and increases the work of breathing Hyperinflation of the lung may also hinder cardiac filling, lead to dyspnea and reduce exercise performance of the patient. The detrimental effects of COPD often lead to a cascade of symptoms that eventually impairs quality of life and increases the risk of death of the patient

In the United States, the term COPD includes two main conditions, which are emphysema and chronic bronchitis. In emphysema, the walls between many of the air sacs are damaged. As a result, the small airways and air sacs lose their structural integrity and the ability to maintain their optimal shape. This damage also can destroy the walls of the air sacs, leading to fewer, but larger air sacs instead of the many small structures found in healthy lung tissue. When this destruction occurs, the amount of gas exchanged by the alveoli of the lungs may be significantly reduced. Within the lung, focal or “diseased” regions of emphysema, characterized by a lack of discernible alveolar walls, are referred to as pulmonary bullae. Within diseased lung, these inelastic pockets (>1 cm in diameter) of dead space do not contribute to gas exchange and are often considered to be primary candidate areas for therapy.

In chronic bronchitis, the lining of the airways becomes inflamed, generally as a result of ongoing irritation. This inflammation results in thickening of the airway lining and the production of a thick mucus, which may coat and eventually congest the airways of the lung. It is common to find patients with COPD having symptoms of both emphysema and chronic bronchitis.

COPD is a major cause of disability and is the third leading cause of death in the United States. Millions of people are diagnosed with COPD. Many more may have the disease and may be unaware of the progression of the disease, as COPD develops slowly, such as over the course of many years. Symptoms often worsen over time and can limit the ability to do routine activities. Severe COPD may prevent a patient from doing even basic activities like walking, climbing stairs, or taking care of oneself Currently, there is no cure to COPD, and while research is ongoing, current medical techniques offer no solution for reversing the damage to the airways and lungs associated with the disease.

Fortunately, there are treatments and lifestyle changes can help reduce the symptoms of COPD, allow patients to stay more active, and slow the progress of the disease. Reducing the increase of risk of COPD from smoking is considered to be the most effective lifestyle change. As a more drastic approach, one treatment that temporarily addresses the symptoms of COPD is Lung Volume Reduction Surgery (LVRS), which surgically removes poorly functioning portions a lung (typically up to twenty to thirty-five percent). By removing relatively diseased portions of a lung, LVRS reduces the overall size of the lung and opens the volume within the chest for the remaining lung to expand and contract. The remaining lung is elastic and able to expand into the newly opened volume of the chest. LVRS improves the capacity of the lung to breath by allowing the remaining portion of lung to expand and contract to a greater extent than before LVRS. Thus the remaining lung has an enhanced capacity to take in air and exchange gases. The obvious drawback is that LVRS is highly invasive and requires open-lung surgery, rendering it only a last-resort option for many patients.

Although LVRS has benefits, as compared to other optimized medical therapy, the risks and mortality/morbidity rates of LVRS require serious consideration before surgery. Up to twenty eight percent (28%) of patients have been reported to need in-hospital stay for rehabilitation facilities for one (1) month or more after surgery. Main factors of LVRS-related morbidity include adverse effects of general anesthesia during surgery, mechanical ventilation during surgery and the fragile clinical status of patients with advanced emphysema. That said, conceptually, the removal (resulting in the overall reduction) of emphysematous lung tissue in LVRS increases the available volume in the chest cavity, within which the remaining portion of the lung may expand. The greater expansion of the remaining lung tissue stretches the tissue to a greater extent than the tissue expanded before LVRS. By effectively restoring the elastic recoil of the lung tissue in some parts of the lung, airway traction is at least temporarily improved and the symptoms of airway closure within the lung may be delayed significantly.

To achieve the benefits of LVRS with a lower morbidity rate and length of recovery/hospital stays, the minimally invasive techniques and devices have been developed, with varying degrees of success. These techniques may include inserting, deploying and activating lung reducing devices with a lung via the trachea of the patient. These techniques do not require an open, surgical approach, and are envisioned to require minimal general anesthesia (or only a reduced period of general anesthesia or conscious sedation). Recovery time and hospital stays that result from these minimally-invasive devices, applications or techniques would also be dramatically reduced as compared to LVRS.

Examples of less invasive devices and techniques for lung volume reduction are shown in U.S. Pat. Nos. 6,599,311, 7,128,747 and 8,157,837, and in Kontogianni, “BRONCHOSCOPIC NITINOL COIL LUNG REDUCTION DEVICEATION: A NEW LUNG VOLUME REDUCTION STRATEGY IN COPD”. Respiratory, EMJ European Medical Journal, p. 72-78 (October 2013). The lung reduction coils are deployed to fasten primarily to poorly performing regions of the lung. As the devices expand, bend, retract or otherwise change shape, they seize the attached portion of the lung and physically compact lung tissue. This action collapses the lung tissue affixed to the device, as well as additional tissue along the path of the device, surrounding the thereby reduce the overall size (and volume) of the lung similar to LVRS.

While the above-mentioned devices and methods and traditional LVRS demonstrate that there is a basic correlation between a reduction in unhealthy lung volume and improvements in patients suffering from emphysema, the current limitations of these approaches suggests that vast improvements are yet to be made in order to fulfill a need in the current state of the art.

### SUMMARY OF INVENTION

Minimally invasive surgical techniques for lung reduction and lung reducing devices have been shown, at times, to be effective in human patients. The devices have yet to enter into widespread use. While the lack of use is at least partially due to the lack of government approval in the United States, it is posited that existing lung reducing devices and the techniques to implant the device do not represent optimal solutions for lung reduction. The inventors have identified a need for lung reducing devices that are safe, easy to deploy, reliable and capable of cumulatively collapsing large portions of a lung, for example at least fifteen to twenty percent (>15-20%) of the overall volume of the lung.

The inventors have conceived of and disclose herein, implantable lung volume reducing devices and medical techniques for implanting lung volume reduction devices through the trachea and bronchi, using minimally-invasive deployment and surgical techniques. The lung reducing devices may be used to reduce the volume of one or more lung, thereby increasing the elastic recoil of the remaining lung volume.

These devices may also delay closure of the small airways in the lung during a breath and lower the Residual Volume (RV) in the lung. A reduction in RV results in less air trapped in the lung at the end of each breath and suppresses hyperinflation of the lung. These improvements to lung dynamics may contribute to a reduced strain in breathing and a reduced sense of dyspnea.

RV is an accepted index of disease severity and the benefit of a lung reduction therapy is generally accepted to be proportional to the reduction in RV. Reduced thoracic gas compression and improved expiratory flow may translate to an improvement in chest wall and diaphragm configuration and mechanics, reduced dynamic hyperinflation and strain of breathing, and better cardiac performance.

A novel treatment is disclosed herein for patients suffering from COPD comprising the application of a minimally invasive bronchoscopic technique to implant a lung reduction device into a lung airway of a patient. The implantable lung reduction device, which may be generally referred to as a “clip” roughly comprises two or more distal arms that are envisioned to span adjacent airways. The arms of the device are connected or joined at a device body immediately upstream of the bifurcation (also referred to as a “fork”) in the airway, with the device body defined beginning from the upstream (proximal) end of the device through to the distal-most intersection of the arms or the device saddle. The tissue separating the two adjacent airways immediately downstream of the airway junction may be referred to as the airway septum. The device may bias the tissue of the septum together to affect the airway passages and the overall lung volume downstream of the bifurcation. The biasing of the tissue compressed and collapses, at least partially, the lung tissue in the vicinity of the adjacent airways and the bifurcation. The overall lung volume is reduced due to the local tissue collapse. Implanting several devices (i.e. 10, 15, 20 or more), implanting devices within a single lobe, or staging delivery of lung reduction devices provides a cumulative reduction that may amount to 10%, 20% or more of the volume of the lung.

The lung reduction device configured for delivery may be a clip, fork, clamp, clasp, pin device or other device. In some embodiment, it is envisioned that the device may be dimensioned to pass within a channel along the interior of a delivery device. The working channel of a scope dimensioned and configured for passage through or directly within the trachea 10 may be used within such a delivery system. The device is further configured to, when deployed, collapse two or more downstream branches of the lung airway by biasing the branches towards one another. One challenge that faces the use of implantable lung reduction devices is the positioning and delivery of the device. It is envisioned that procedures involving lung reduction device delivery to a lung directly through the trachea or with a modified bronchoscope would be minimally invasive, reliably safe and would have the potential to become the preferred procedure to treat COPD. At least one such delivery system for a lung volume reduction device is described herein. The delivery system safely delivers the lung reduction device to the diseased portion(s) of the lung.

In at least one aspect, a bronchoscope delivery system may be inserted through the trachea to insert the lung reduction device(s) into the lung airway. The lung reduction device may be deployed using a standard, adapted or modified bronchoscope. A bronchoscope is a device used to pass through the trachea and inspect the lung parenchyma or pleural space. Bronchoscope procedures are common, minimally invasive and safe. In some instances, it may be further advantageous to perform bronchoscopic delivery, as some practitioners prefer one or more systems of feedback to assist in the delivery process. The bronchoscope may be a carrier for a sterile and disposable delivery system for the lung reduction device. The delivery system may further comprise a catheter, a guide wire, and a mechanism for delivery and deployment and possibly retrieval of the lung reduction device(s).

In at least one further aspect, the delivery system may include a guide wire, a catheter and a delivery tool at a distal end of the catheter. The guide wire serves as a specialized guide for the catheter, which is used by the surgeon to identify and select a pathway through the lung airway and bifurcations in the lung airways to treat. The movement of the guide wire through the airways may be viewed on display screens connect to X-ray fluoroscopy device or computed tomography scanners (CT scanners) imaging the chest of the patient. The guide wires also support the catheter, as the catheter is maneuvered through lung airways to the selected bifurcation. The guide wire may also be used to help determine the appropriate length of the lung reduction device.

In at least one aspect, a catheter functions as a conduit to deliver the lung reduction device from outside the patient to the targeted treatment area. The catheter can also be used to re-position or remove the lung reduction device. The lung reduction device may be removed by reversing the methods of the deployment procedure Alternatively, in at least one aspect, the lung reduction device may be retracted into a tubular sheath that would be extended from the catheter, and the catheter/sheath combination may then be safely retracted from the airway possibly through the working channel of the bronchoscope.

Using a wide range of medical imaging techniques suitable for the chest cavity and lung, a physician may select an airway bifurcation, as a target to approximately seat the device body of lung reduction device. The distal end of the delivery system may be loaded to contain the lung reduction device and is subsequently extended towards the selected airway bifurcation in the downstream direction. The lung reduction device may have a device body connected to at least two arms that extend distally away from said device body. The device may have a reduced profile for delivery, wherein in the reduced profile the device is capable of being advanced downstream or distally into a small airway of the lung. The device may also be configured to assume an expanded profile once deployed within the airways of the lung, wherein the expanded profile the device secures to lung tissue.

Sufficiently reducing lung volume in at least one or more lobes in the lung is critical to the operation of the device. The dimension and configuration of the device must be advantageous for both delivery and use in collapsing unhealthy lung tissue. Generally, the device body comprises a stem, which may be configured to face upstream and interact, as necessary, with the delivery tool of the delivery system (e.g. a grasper, or pusher or other device that may be extended from a bronchoscope). Opposite the stem, the device body comprises a vertex that may be configured to face in the downstream direction. The profile of the device is generally affected by the shape and angle at the vertex of the body. At the vertex of the body, the device body splits into its respective distally-extended arms. The device may further comprise a saddle at or near the vertex of the device body. In some instances, the saddle may also be located at the vertex, but in more complex configurations the saddle and vertex may be separate from the other. The saddle be suitable and specifically configured for extended contact with the septum, the tissue immediately downstream of the airway bifurcation. In more complex devices, the saddle is found immediately downstream of the distal-most intersection of the arms. Alternatively, when the device or devices are placed, the saddle is located immediately upstream of a respective septum.

The tissue of the septum at or near the bifurcation may serve as both a target for device delivery and/or a physical stoppage and fixation point for the lung reduction device, as access to the area can be visually confirmed and where the tissue therein is relatively devoid of blood vessels. These tissue characteristics may help to reduce injury, inflammation, bleeding and other risks associated with implantation, which are increased, as result of the bias imparted by the arms of the device. The device may be seated on the saddle of the device, within the lung airway immediately upstream or adjacent to the tissue of the septum. With the device body and saddle seated over the septum 28 and with the arms positioned within the branches, the lung reduction device may elastically bias the airways in the direction of the other arm, narrowing the lung tissue held between the airways containing the arms. In alternate embodiments, further manipulation of the device may be needed to create the appropriate biasing force needed to close the arms and compact their respective airways.

A novel method is disclosed for minimally or non-invasively reducing the volume of one or more hyperinflated lungs, and improving the pulmonary function of a patient with chronic obstructive pulmonary disease, including through reducing the volume of one or more hyperinflated lungs, removing trapped or residual air from the lung and increasing the metabolic efficiency of the thoracic diaphragm. Diseased lung tissue is frequently made of the inelastic pockets that both contain significant portions of trapped air and lack the ability to contribute to gas exchange and may be identified using various medical imaging techniques to direct therapy to candidate lung areas for therapy.

Based on patient need, practitioner preference, or other factors, the lung reduction device, delivery device and methods may be selected from one or more of the alternative lung reduction devices and methods of use described herein. For example, a surgeon may be presented with lung reduction devices of various sizes, e.g., length of the legs, and biasing force (force applied by the clip to close the legs of the clip). The surgeon implants each of the selected devices during the course of a lung reduction surgery.

A novel method is disclosed to reduce the size of a lung comprising: inserting a bronchoscope into the patient airway, advancing the bronchoscope distally into the patient lung, identifying disease/targeted tissue or the airways leading to the targeted area of lung parenchyma, and navigating the bronchoscope to the selected airway. Once placed, the catheter and guide wire may be sequentially placed into the working channel of the bronchoscope, advancing the guide wire out of the working channel and into the targeted airway, holding the guide wire fixed relative to the bronchoscope and advancing the catheter distally as far as possible but generally not past the tip of the guide wire, possibly removing the guide wire from the catheter while maintaining the catheter position, abutting the proximal end (e.g. stem) of the lung reduction device with the delivery mechanism (e.g. a delivery tool, grasper or gripper), inserting the lung reduction device in the delivery configuration into the catheter and by advancing the delivery tool and the lung reduction device, positioning the lung reduction device into the target airway and deploying the lung reduction device, and further verifying the position of the lung reduction device prior to releasing the lung reduction device. A delivery tool may be coupled to, contact, or abut the proximal end (e.g. the stem) of the lung reduction device to deliver it through the catheter providing control of delivery and deployment of the lung reduction device. The guide wire and the catheter may continue to be used to deploy additional lung reduction devices.

In some embodiments, interaction with the bronchoscope wall, an optional delivery sheath, other feature of the delivery device, or environmental features (e.g. tissue or airway walls) may spread the arms of the device during delivery. In one embodiment, the walls of the septum force open the arms of the device, allowing the arms to advance downstream into lung airways. The tissue between the airways is compressed by the arms of the device. Alternatively, the delivery sheath may be retracted to allow the arms to separate controllably to allow for positioning over the septum 28. With the arms separated, the device is advanced downstream into the lung airway to further position arms within the branches of the bifurcated airway(s). The device may employ additional features to increase the bias of the arms following the positioning of the device. The catheter, delivery features and/or other devices used to position the lung reduction device are completely removed after fully positioning the lung reduction device.

In some embodiments, as each generation (branch) of airways generally decrease in diameter at it extends distally into the lung, the method may further comprise selecting bronchoscope and catheter with diameters sufficiently narrow to navigate the patient airway up to, but not beyond the septum of the target area. By advancing the delivery devices to the airway just upstream of the target area and septum, the device may be delivered using the septum as a both visual guide and physical barrier, drastically increasing the potential speed of device placement, while also mitigating many of the risks of device implantation.

Several lung reduction devices may be implanted throughout the patient lung or lungs, targeting one or more pairs of airways at each point. The combined effect of the lung reduction devices is to collapse a large portion (e.g., ten (10%) to thirty (30%) percent of the lung). In some instances, the lung reduction device may be manipulated by a handle, which is grasped and released by a surgical insertion tool, such as a tool introduced through the distal end of a bronchoscope or catheter. The lung reduction devices may be supplied in different sizes, each of which may have a different length of the arms or compression strength. The different sizes may be selected to accommodate anatomical variations of airways, or to restrict entry to specific generations of airway. The lung reduction device may be designed to be biocompatible, atraumatic and configured to remain implanted in the small airways of the lung for extended periods of time.

The intended physiological benefit of the lung reduction devices is similar to the desired effect resulting from LVRS, which is to reduce the volume of a lung by collapsing regions of the lung tissue (parenchyma) that are diseased and are not effectively exchanging gases between air and blood. Lung reduction is achieved by the lung reduction devices bringing the branches of the airway closer together and compressing the diseased lung parenchyma between the airway branches. The lung reduction devices may also apply tension to relatively healthy, and well-functioning lung tissue near the collapsed airways. This increase in tension may help to increase the elastic recoil of the remaining lung tissue. Also, collapsing diseased lung tissue redirects air in the lung to healthier portions of the lung.

As a result of volume reduction in the lung, small airway closure may be delayed during expiration, may occur at lower RV, and may result in less air trapping. A reduction in RV subsequently results in less hyperinflation. This cascade may contribute to increasing the efficiency of breathing and reduced common symptoms of lung hyperinflation, specifically the sensation of shortness of breath (dyspnea). This therapy may target specific and local diseased regions of the lung, which in some cases may be identified by imaging, but may also be considered for use in treating the symptoms of a homogeneous emphysema, wherein most of the lung is affected. It is expected that one or more than one lung reduction devices may be necessary to achieve adequate therapeutic effects and that such devices can be added and removed, as needed.

In accordance with these and the further aspects of the present invention, a method and device is described for reducing the volume of a lung. The present invention provides advantages of a minimally invasive procedure for alleviating at least some of the symptoms associated with COPD and emphysema without the risk and complications associated with conventional LVRS surgery.

### BRIEF DESCRIPTION OF THE DRAWINGS

Other advantages of this invention are made apparent in the following descriptions taken in conjunction with the provided drawings wherein are set forth, by way of illustration and example, certain exemplary embodiments of the present invention wherein:

FIG. 1A is a schematic representation of normal, healthy airways of the human body;

FIG. 1B is an enlarged view of the portion within the dotted line circle of FIG. 1A and shows healthy air sacs and bronchi leading to the sacs

FIG. 1C is a schematic representation of hyperinflated lungs indicative of COPD

FIG. 1D is an enlarged view of the portion of FIG. 1C within the dotted line circle of FIG. 1C and shows diseased air sacs due to COPD.

FIG. 2 is a schematic diagram representing measurement of lung volumes under normal lung function.

FIG. 3A is a table that shows the divisions of airways based on Weibel's observation airway branching.

FIG. 3B is a partial anatomical diagram showing the divisions of airways on a single linear branch of the lung down to the final generation and alveoli

FIG. 4 is a schematic diagram of a lung volume reduction device in the process of device delivery.

FIG. 5A is a schematic illustration of a lung volume reduction lung device in a delivery configuration prior to deployment.

FIG. 5B is a schematic illustration of the deployment process of a lung volume reduction device.

FIG. 5C is a schematic illustration of a deployed lung volume reduction device.

FIG. 6A is a schematic illustration of a lung volume reduction lung device in a delivery configuration prior to deployment.

FIG. 6B is a schematic illustration of the deployment process of a lung volume reduction device.

FIG. 6C is a schematic illustration of a deployed lung volume reduction device.

FIG. 7 is a top view of a lung volume reduction device showing one possible interaction with an activating locking mechanism.

FIG. 8A is a perspective view of a lung volume reduction device at neutral or rest with arms in an open or variable configuration, prior to device deployment.

FIG. 8B is perspective view of a lung volume reduction device at rest with arms in an open or variable configuration, partially interacting with one possible activating locking mechanism.

FIG. 8C is perspective view of a lung volume reduction device at rest with arms in an open or variable configuration, further interacting with an activating locking mechanism.

FIG. 9A is a side elevation view of the lung volume reduction device of FIG. 8A.

FIG. 9B is a side elevation view of the lung volume reduction device of FIG. 8B.

FIG. 9C is a side elevation view of the lung volume reduction device of FIG. 8C.

FIG. 10A is a schematic representation of a diseased lung.

FIG. 10B is a schematic representation shown a scaled-up lung volume reduction device deployed and the resulting lung volume reduction.

FIG. 11 is a flowchart showing a logic diagram that may be used, in part, to make one or more determinations as part of a method of lung volume reduction.

FIG. 12A is a side elevation view of a lung volume reduction device that can be rotated from the vertex to engage the device arms shown with an optional collar locking mechanism.

FIG. 12B is a side elevation view of a lung volume reduction device that can be rotated from the vertex to engage the device arms shown with an optional collar locking mechanism.

FIG. 12C is a side elevation view of the lung volume reduction device of FIG. 10A, partially engaged shown with an optional collar locking mechanism.

FIG. 12D is a side elevation view of the lung volume reduction device of FIG. 10A, fully engaged in a locked position shown with an optional collar locking mechanism.

FIG. 13 is a schematic representation of a bronchoscope within an airway of a lung, equipped with camera and catheter/guide wire components and fitted within a working channel

FIG. 14A is a detailed view and schematic representation of an airway clip being positioned and subsequently deployed

FIG. 14B is a detailed view and schematic representation of an airway clip being positioned and subsequently deployed using graspers or forceps.

FIG. 15A is a top or overhead view of two bifurcating airways showing a clip device that is oriented such that the arms of the device are not entering two parallel airways and in a way that requires repositioning.

FIG. 15B is a top or overhead view of two bifurcating airways showing a clip device that is oriented such that the arms of the device are entering two airways in parallel.

FIG. 16A is a partial views of the proximal device body an airway clip showing the clip with a notched device body.

FIG. 16B-C shows a grooved channel that may be adapted for delivery of an airway clip.

FIG. 16D-E shows a grooved channel with an airway clip positioned within the channel

FIG. 17 shows at least two clips positioned in sequence for positioning and subsequent delivery in a catheter.

FIG. 18A-D show a sequence of three clip as they are positioned and subsequently deployed with the use of a single guide wire.

FIG. 19A shows a clip being delivered from a catheter using a dual guide wire approach.

FIG. 19B shows a clip positioned over the septum of an airway bifurcation using dual guide wire approach.

FIGS. 20A and B show an alternate delivery mechanism with the device loaded onto the exterior of a bronchoscope.

FIGS. 21A and B show an alternate delivery mechanism with the use of one or more guide wires, with the device loaded onto the exterior of a bronchoscope.

### DETAILED DESCRIPTION OF THE INVENTION

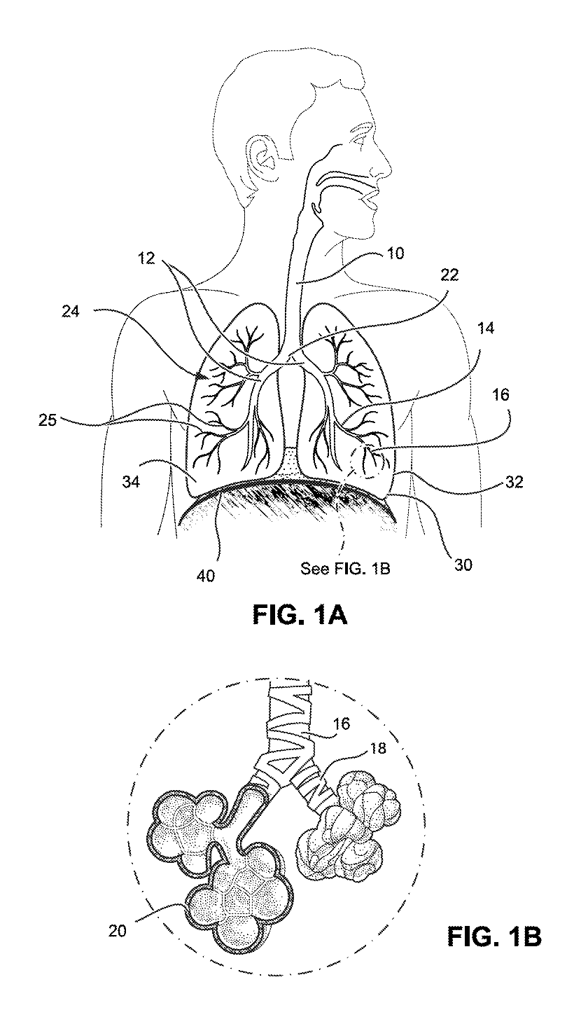

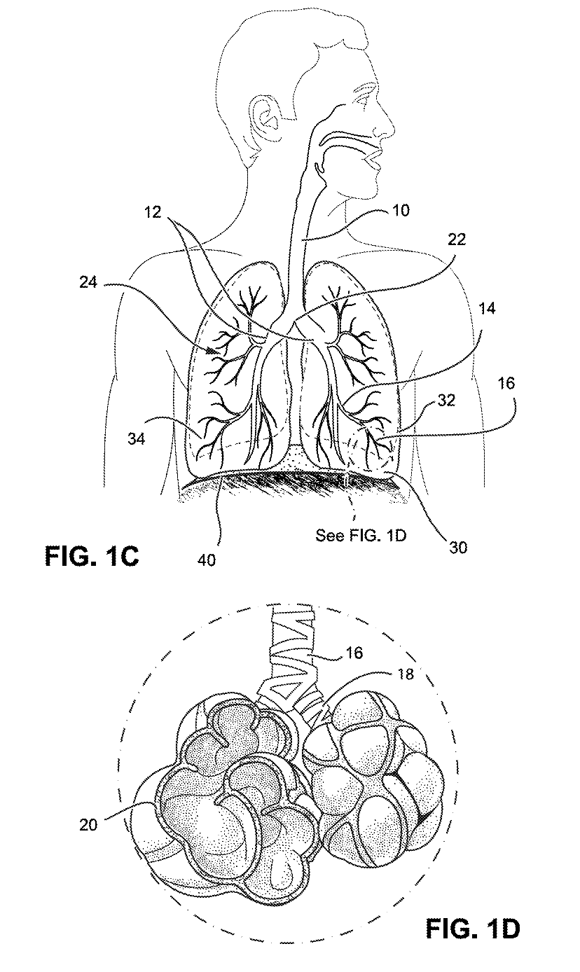

FIGS. 1A, 1B, 1C and 1D illustrate the respiratory system located primarily within the thoracic cavity. In human beings, the lungs 30 are present in pairs and are located in the pleural cavities of the thorax on either side of the heart. The lungs are separated from the abdominal cavity by the muscular thoracic diaphragm 40, which expands and contracts to facilitate breathing The lungs 30 of a typical adult human are about 25 to 30 cm long and are approximately cone shaped. A protective membrane, called the pulmonary or visceral pleura, protects the lungs and separates each lung from the parietal pleura, which covers the chest wall, by the thin layer of pleural fluid. The lungs are separated by the mediastinum, which contains the heart, trachea, esophagus and blood vessels. The lungs normally have clear anatomical divisions known as lobes. The right lung 34 is divided into three lobes called superior, middle and inferior lobes, by the oblique and horizontal fissures that are folds of the visceral pleura. The left lung 32, which is slightly smaller, is divided into two superior and inferior lobes, by the oblique fissure.

Air inhaled from the environment initially enters through the mouth or nose, passes the larynx, and is carried down through the trachea 10 (or the wind pipe) into the lungs 30. The conducting airways of the lungs begin at the tracheal bifurcation 22. The lung airways 24, which are long tubular structures that conduct air through the respiratory tract, include the first generation (or primary) bronchi 12, commonly known as the right or left bronchus, that lead air into each of the lungs where they subdivide into secondary bronchi 14. Each second generation (or secondary) bronchus 14 then leads into a single lobe where it subdivides further into tertiary bronchi 16. These tertiary bronchi lead into each of the pyramid-shaped bronchopulmonary segments (not shown), which are separated from one another by connective tissue septa. Each of these bronchopulmonary segments supplied by bronchi 12, 14, 16 is served by a corresponding artery and vein. Blood supply to these segments is clinically important as pulmonary disease is often confined to one or a few unhealthy segments, which can be treated (e.g. surgically removed, compressed, or otherwise reduced in volume) with minimal effect on the overall function of the remaining healthy segments.

Within the bronchopulmonary segments, branches 25 of the airway 24 divide from the tertiary bronchi in several generations of numerous smaller bronchioles 18. Weibel (1963) observed twenty three (23) successive branches 25 of conducting airways 24 ranging from the trachea 10 through to the terminal bronchioles in the normal human respiratory system. The branches 25 of the conducting airways 24 lead into the respiratory zone of the lungs, which are comprised of respiratory bronchioles, alveolar ducts and alveoli 20. The alveolar ducts lead into a terminal branch or into alveolar sacs, clusters of individual microscopic structures known as alveoli 20 (see details of FIGS. 1B and 1D). Within the respiratory zone, the alveoli 20, thin-walled sacs that allow air passage into the lungs, act together to form a respiratory surface for the lung. It has been estimated that there are approximately 500 million alveolar sacs present in a human lung. The sheer number of these small, thin-walled alveoli 20 functioning in unison, is able achieve an enormous surface area for gas exchange, roughly 50-100 square meters.

As a person breathes in air from the environment, the alveoli 20 stretch, drawing oxygen in and transporting it into the blood. Simultaneously, carbon dioxide is removed from the blood. During the process of exhalation, the alveoli contract, forcing carbon dioxide out of the body. To optimally perform their function, alveoli 20 must maintain their expandable surface area, structural integrity and overall elasticity. Emphysema is a condition that involves damage to the walls of the alveoli of the lung. In emphysematous lungs, the alveoli and lung tissue are gradually destroyed. As the disease progresses, the walls separating the alveoli are reduced, resulting in a loss of surface area and elasticity diminishing the ability of the lung parenchyma to properly support airways 24. Bronchioles eventually collapse and cause an obstruction to exhalation, which traps air inside the alveoli. FIG. 1B illustrates the breakdown of the walls of the alveoli in emphysema, which causes a decrease in respiratory function and breathlessness.

While different muscles groups contribute to inhaling and exhaling, the largest and most efficient muscle that plays a role in breathing is the thoracic diaphragm, known simply as the diaphragm. The diaphragm is a large muscle that lies under the lungs 30 and separates them from the organs of the abdominal cavity below, such as the stomach, intestines, liver, etc. As the dome-shaped diaphragm 40 contracts, it moves down (descends) like a piston in a cylinder, it flattens, the ribs flare outward, the lungs expand and air is drawn in through the airways 24. This process is called inhalation or inspiration. As the diaphragm relaxes, the lung 30 contract to their original position expelling air from the system urged by elastic recoil of the lung tissue. This is called exhalation or expiration. The lungs, like balloons, require energy to expand but no energy, other than the stored energy of elastic recoil, is normally needed to let air out. Additional muscles that are used in breathing are located between the ribs (e.g. intercostal muscles) and among certain muscles extending from the neck to the upper ribs. The diaphragm, muscles between the ribs and one of the muscles in the neck called the scalene muscle are involved in almost every breath.

FIG. 1A shows the lungs 30 in relation to the diaphragm 40. In the lungs of a healthy individual, as seen in FIG. 1B air passes efficiently through the alveoli and airways 24 of the lung. The diaphragm 40 has an elastic curvature, which is sloped upward at rest and may expand downward to allow inhalation. In some conditions, including emphysema, as a result of air trapped in the hyperinflated lung, the diaphragm may be flattened or pushed down and lose the ability expand and contract optimally. In these instances, the expansion of the lung becomes increasingly dependent on the function of other muscles, which are metabolically efficient compared to the diaphragm. Over time, the brain and body may compensate for this imbalance resulting in sensations of breathlessness or shortness of breath.

FIG. 1C illustrates the flattening of the diagram 40 from lung hyperinflation. In a significant proportion of patients with diseased lung tissue 50 (e.g. resulting from emphysema or other pulmonary disease) reduced lung elastic recoil, sometimes combined with expiratory flow limitation, eventually leads to lung hyperinflation during the course of the disease. The alveoli 20 and small airways 24 of the lung may lose their shape and ability to move air efficiently (see FIG. 1D) and can lead to an inability to exhale fully. When this occurs, the lung can be hyperinflated at rest (static hyperinflation) and/or during exercise (dynamic hyperinflation) when ventilation requirements are increased, breathing rate is accelerated and expiratory time is shortened. Ultimately, diaphragm fatigue, resulting from the inefficient shape of the muscle, can lead to the reduced and eventual inability to breathe. The progression of hyperinflation is clinically relevant for patients with emphysema as mainly because it contributes to the dyspnea and morbidity associated with the disease. In dire situations, patient may be placed on a mechanical ventilator, as a life saving measure. Both quality of life and life expectancy for patients with severe conditions, including late-stage emphysema, is extremely low with fewer than half of patients surviving an addition 5 years.

To help monitor the health and function of the lungs, as well as the progression of deterioration or disease states, the evaluation of lung volumes provides a tool for understanding the changes that may occur in lung mechanics The breathing cycle is initiated by expansion of the chest. Contraction of the diaphragm causes it to flatten and move downward. If chest muscles are used, the ribs expand outward. The resulting increase in chest volume creates a negative pressure that draws air in through the nose and mouth. Normal exhalation is passive, resulting in recoil of the chest wall, diaphragm, and lung tissue.

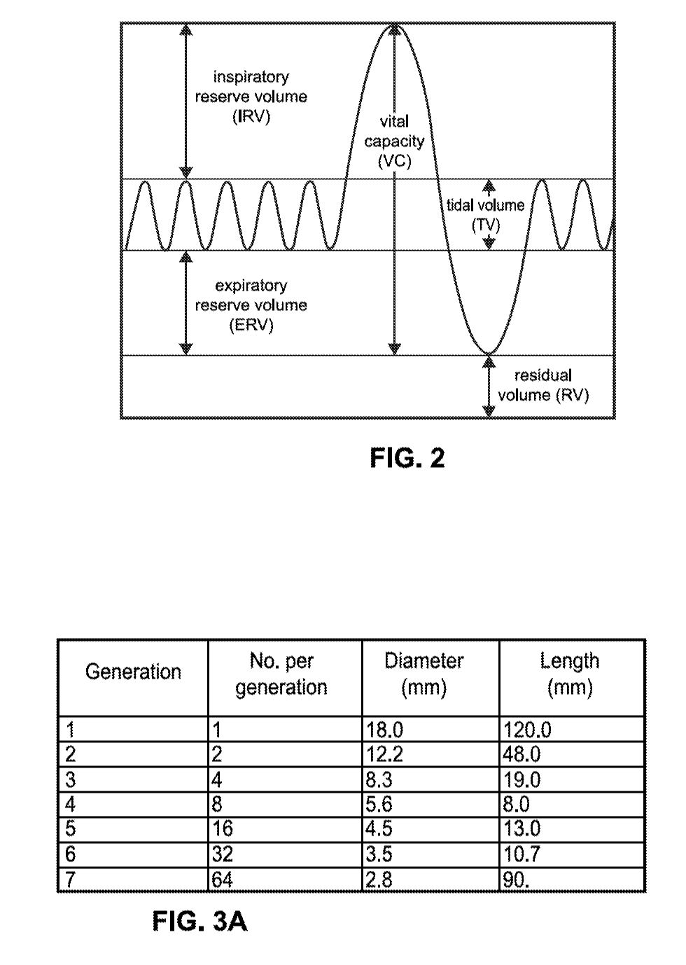

FIG. 2 illustrates normal breathing at rest during which approximately one-tenth of the total lung capacity is used. Greater amounts are used as needed (i.e., with exercise). Tidal Volume (TV) is the volume of air breathed in and out without conscious effort. The additional volume of air that can be exhaled with maximum effort after a normal inspiration is Inspiratory Reserve Volume (IRV). The additional volume of air that can be forcibly exhaled after normal exhalation is Expiratory Reserve Volume (ERV). The total volume of air that can be exhaled after a maximum inhalation is Vital Capacity (VC). VC equals the sum of the TV, IRV, and ERV. Residual Volume (RV) is the volume of air remaining in the lungs after maximum exhalation. The lungs can never be completely emptied. The Total Lung Capacity (TLC) is the sum of the VC and RV. Evaluation of lung function may be used to determine a patient's eligibility for therapy as well as to determine successful treatment with the described invention.

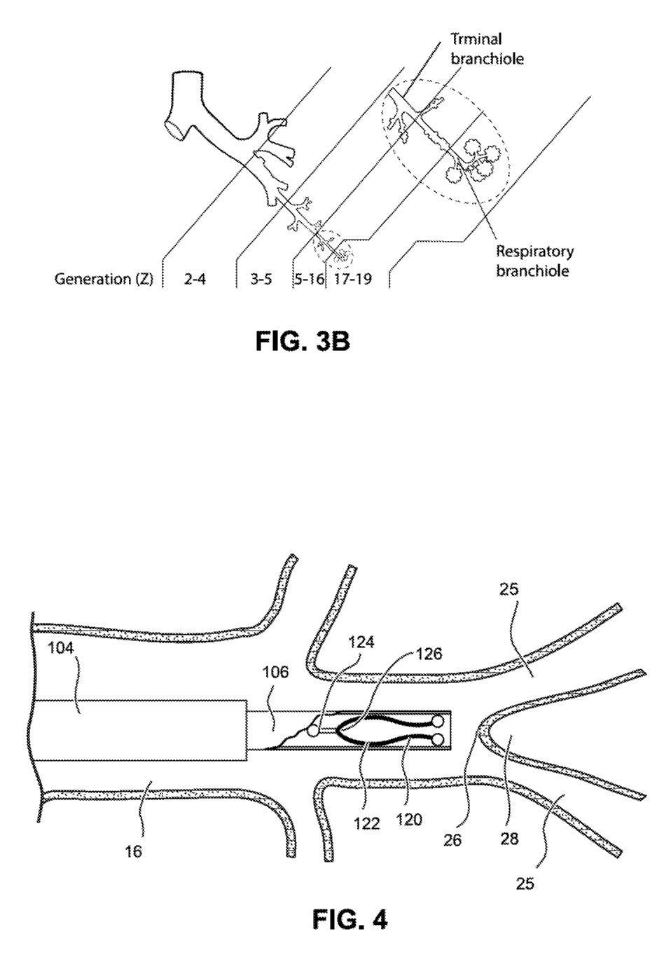

Table of FIG. 3A shows the relevant divisions of airways and is an illustration model based the Weibel's observation of the airway 24 branching. FIG. 3B shows a single continuous pathway from the first airway bifurcation at the carina through to the two smallest generations.

The terminal bronchioles are only a single generation removed from respirator bronchioles, which lead directly to the alveolar duct and alveoli 20. The generations of interest for delivery and use of the device may be the intermediate generations. For example, according to these observation, up to eight lung reduction devices can be inserted into the distinct branches of 4^{th} generation airway as it splits into the 5^{th} generation. The use of these device in a single unhealthy lobe of the lung would decrease the volume of that lobe, allowing the remaining healthy portions of the lung to function more efficiently.

A lung reduction device to be implanted into the divisions of the lung may be dimensioned to access at least the 5^{th}, 6^{th} or 7^{th} generation of the lung. The distal ends of the devices may be tapered, in some embodiments, to accommodate natural narrowing of the airways towards distal end. These distal ends, in the form of arms extending away from a device body, can be narrow in diameter near the device body (e.g. narrower than the diameter of the preceding generation, less than 2 mm, or less than 1 mm) and vary in length from approximately 10-3 mm long.

The devices and methods of providing a minimally invasive lung volume reduction system allows for a treatment option that is available to patients suffering from late-stage pulmonary disease and emphysema. A lung volume reduction system may comprise a lung reduction device designed to be delivered to a lung airway 24 of a patient in a delivery configuration and deployed to compress unhealthy lung tissue 50, thereby improving the function of the remaining healthy tissue.

FIG. 4 illustrates the general concept behind a lung volume reduction device in the form of a clip. The clip is brought to a target location with tissue 50 near an airway bifurcation 26 (also referred to as a septum or fork), preferably using minimally invasive techniques. The particular bifurcation may be chosen based on the severity of the disease in that lobes or region of the lung and is adjacent to branches 25 that directly lead to damaged bronchioles or alveolar sacs. The arms 122 of the device may bias unhealthy or diseased tissue 50 between the branches 25 toward one another to affect the septum 28 (the tissue separation the two adjacent airways immediately downstream of an airway junction) caught in between the arms 122 of the device to affect overall lung volume downstream of the bifurcation.

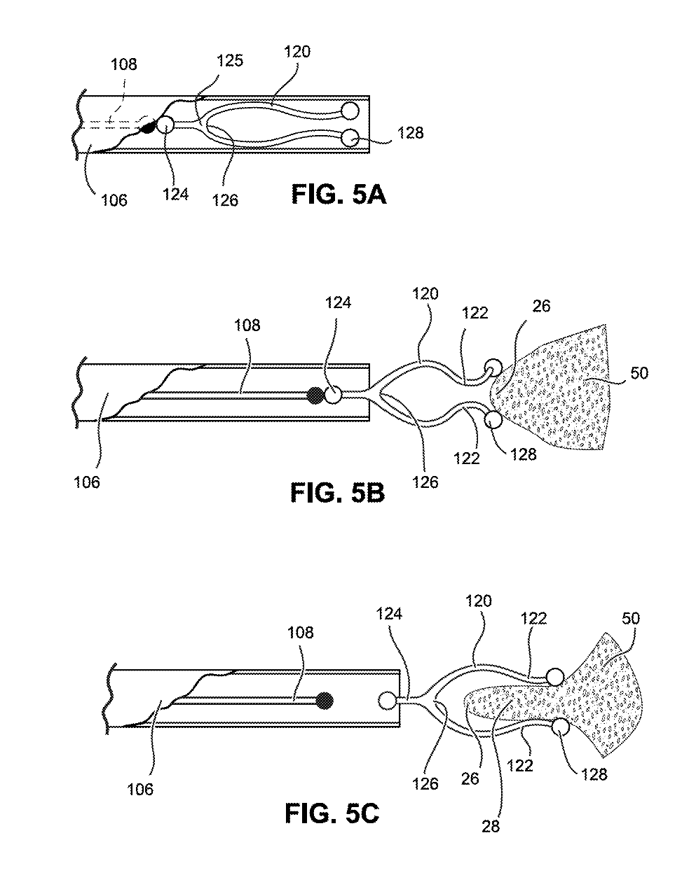

FIG. 5A-C show a device being position and deployed in a manner that may reduce volume once the clip is advanced to its final position. The lung reduction device 120 includes a stem 124 at a proximal end, a vertex 125 and a saddle 126 (at the distal edge of the bifurcation of the device) which connects two or more arms 122 that extend distally downstream from stem 124. The aims 122 may be biased towards a closed position, which assists in collapsing tissue between braches of the lung airways into which the arms are inserted. The arms 122 are elastically deformable and may terminate at distal nubs 128. The arms may be splayed apart by slidably advancing the device forward to be inserted into branches adjacent the selected airway bifurcation 26 (also referred to as a septum or fork) using the septum 28 of the bifurcation as a guide. The saddle 126 of the lung reduction device forms a joint for the arms and is positioned immediately upstream or adjacent the selected bifurcation 26 in the lung airways 24.

The stem 124 of the lung reduction device supports an optionally rounded proximal end, which is abutted or gripped by a delivery tool 108. The delivery tool 108, e.g., rod, graspers or gripper, may affect the device body at the proximal end, at the rounded end of the stem 124, of the lung reduction device 120. The contact with the proximal end of the device at least facilitates advancement of the lung reduction device into the catheter and into the airway 24. In its simplest form, the delivery tool 108 may be a rod or shaft extending through the catheter 106 and may be attached to a distal end of a bronchoscope or extend through a channel inside the bronchoscope within a catheter to a proximal end of the scope outside of the patient accessible to the operator. The delivery tool 108 on the distal end of the delivery device may close to clamp onto the rounded proximal end of the lung reduction device and open to release the stem 124.

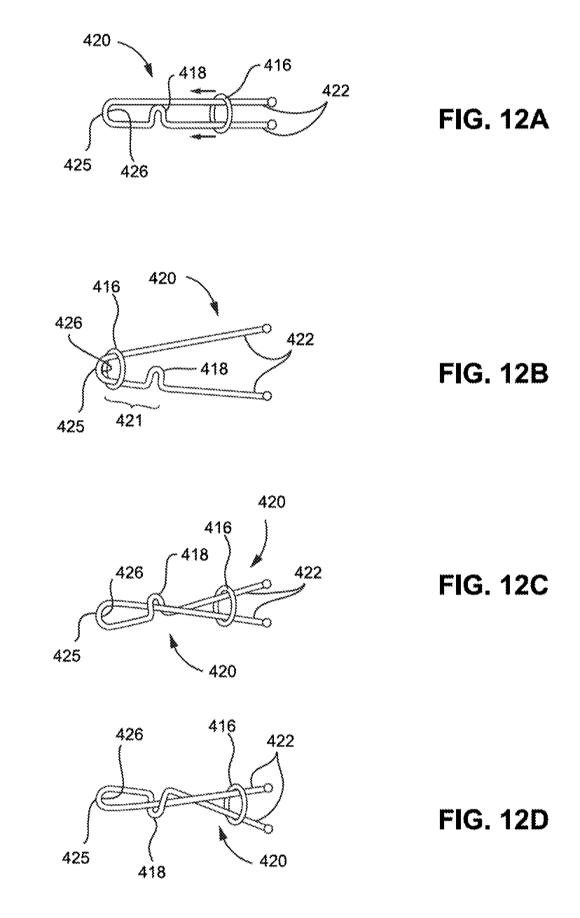

It is envisioned in at least one embodiment that a collar, slideably received and surrounding both arms of the device, may be advantageous in both the delivery and use of the device. The collar 116 (not shown in FIG. 5A-C, but visible in FIG. 12A-D) may be positioned along the length of the device. The collar 116 may affect the shape of the device by compacting of the device arms 122 and body 121 by alternating the positioning of the collar 116 along this length. It is envisioned, for instance, that the collar 116 may be positioned distally during device delivery to surround the device arms 122 and reduce the profile of the device for advancement. In some instances, the shape of the collar 116 may prevent it from being positioned beyond the distal arms of the device. Further, it is envisioned that the collar may be shifted proximally, as needed, to release the arms 122 in order to maximize the volume of tissue 50 captured by the device. It is further envisioned that in at least one embodiment, the delivery tool 108, may be used to reposition the collar 116 along the length of the device, as desired. This fine control of device position and shape may provide a level of maneuverability that may lead to greater favorable results from the use of the device.

Alternatively, the release or delivery of the device may be actuated by sliding an optionally delivery sheath distally from the delivery tool 108 and onto the saddle of the lung reduction device. The sleeve may hold the delivery tool 108 closed and the delivery tool 108 may be biased opened such that the delivery tool 108 opens when the sleeve is off the delivery tool 108. If a sheath is used, it may be positioned along the lung reduction device 120 and envelope the lung reduction device 120 from the proximal to the distal end. The sheath may also envelope the delivery tool 108 while the lung reduction device is being positioned adjacent the saddle and the arms 122 are closed to collapse the diseased tissue 50 adjacent the septum 28 and between branches of the airway 24.

Once the lung reduction device is positioned with the arms in the branches and the saddle adjacent the selected septum 28, the sleeve is retracted away from the septum 28 past the rounded end of the stem 124 (and allows the delivery tool 108 to be opened and release the stem) and over a proximal end portion of the arms 122 to force the arms to close and thereby collapse the branches. Resulting from the retraction of the sleeve and the release of the delivery tool 108, the lung reduction device is fully implanted and released from contact with the delivery tool and the device has collapsed two or more branches to reduce the volume of the tissue 50 located substantially between these branches.

The arms 122 of the lung reduction device that extend distally may be elastic and biased to alter the shape, orientation and forces exerted by the device (i.e through the device arms) during device delivery or once the device is deployed. FIG. 4 shows one generally envisioned device. As a device may be delivered or deployed in an unbiased, neutral position, each device may also be present in at least two other relative positions. When expanded, the device exerts force between the arms that may be maintained so long as the device remains in an expanded position. Alternatively, when compressed the device exerts force outward from the device arms that may be maintained so long as the device remains in a compressed position. It is further envisioned that some devices may interact internally or with a separate mechanism to reorient from a first to second orientation. From a first to a second orientation, it is envisioned that the biasing force exerting between the arms of the device may be increased and sustained. A specialized locking mechanism may be used to maintain the orientation of the device in a second (bias-increased) orientation.

Maintaining a device in a compressed position may also provide advantages. As the device is reoriented or manipulated to have reduced dimensioned, device delivery may be facilitated by this compact configuration. Once deployed, compressing the device by altering the device configuration or by attaching additional locking mechanisms may increase the biasing force through the arms of the device. In an alternative or in combination, the at least one part of the device delivery process may be favorable under a neutral or expanded position. Contrary to the compressed position, a neutral or expanded position would allow the arms of the device to gather and surround the maximum volume of septum tissue with very little resistance. It is envisioned that a device could utilize all three relative positions to maximize the benefits of each described herein.

In at least one envisioned embodiment, each device has two or more arms. The arms 122 may be made of stainless steel, titanium, nitinol, plastic, ceramic or other implantable and lung compatible material. The material forming the arms and nub may be the same as the material forming the stem. Further, the arms, nubs and stem may be a single piece forming the lung reduction device 120. The arms 122 may be symmetrical along the length of the lung reduction device. The arms in each device may closed together to impart a uniform compression on the branches or the arms 122 may be asymmetrical to conform to the branches and facilitate compact collapsing in the delivery configuration.

In at least one envisioned embodiment, a device comprises a device body with a vertex and a saddle, a first arm connected to the device body, and extending distally away from the device body, and a second arm connected to the device body and extending distally away from the device body, wherein the first and second arms are connected to the device body at the device body vertex, and wherein the device body saddle is immediately distal to the distal-most intersection of the first and second arms.

In at least one envisioned embodiment, the device comprises and implantable medical material for placement in a patient lung along a first and second airways of an airway fork. Specifically, the device comprising a first elongated arm dimensioned to reside along a length of the first airway, a second elongated arm dimensioned to reside along a length of the second airways, and a device body with a proximal and distal end configured to remain at the airway fork, the device body further comprising a proximal vertex connecting the first and second arms. In at least one further envisioned embodiment, at least one arm of the device further comprises a locking mechanism or ridge, said ridge configured to remain proximal to the airway fork. In a further embodiment, the device it is further envisioned that the rotation of the vertex reconfigured the device with said ridge contact the opposite arm, applying a bias along the arms to the lengths of airway sufficient to reduce the volume of the lung associated with the airways.

In some embodiments, each arm 122 may have a diameter 0.5 to 3.0 mm and length of 5 to 50 mm The diameter of the arms may taper in a distal direction of the arms, where the proximal end of the arm 122 is at the saddle 126. The length of the arms 122 may be selected based on the dimensions of the septum 28 and branches into which the lung reduction device is to be implanted. There may be lung reduction devices having arms of different lengths to be placed in a specific pair of branches 25 of lung tissue.

At the time of a lung reduction treatment, the physician may have a set of lung reduction devices of different sizes, diameters and other configurations. The physician may select a lung reduction device based on knowledge of the size of the branches into which the devices are to be implanted and information gained from the images of the branches. Imaging by CT or MRI obtained in advance of the procedure can be used to facilitate the election.

Each lung reduction device may be supplied enveloped in an optional sheath that may be selected by the physician and loaded into a proximal end of the catheter 106. The delivery tool (e.g. rod, grasper or gripper) may abut or grip the proximal end of the stem of the selected lung reduction device by sliding the sheath over the delivery tool 108 of the delivery device. If a sheath is not used, the device can alternatively be loaded directed into the proximal end of the catheter 106 to enter through the working channel 110 or directly in the airway 24.

To reduce tissue damage and inflammation, the distal end of the lung reduction device 120 may be constructed and configured to be atraumatic, specifically at the distal nubs 128 of the device. The nubs 128 of the arms 122 of the lung reduction device 120 may be circular, rounded, spherical, hemispherical or otherwise dimensioned to reduce irritation after prolonged contact and bias again airway 24 walls. In some instances, it may be preferred that beyond the arm 122 and nubs 128, additional or all parts of the lung reduction device 120 are configured to be atraumatic. In the alternative, all or portions of the arms 122 or saddle 126 of the lung reduction device 120 may be configured to achieve and maintain contact with the airway 24 between the arms 122. In another alternative, the nubs 128 of the device may be configured to achieve and maintain the inward tension between the arms 122 of the device 120. In another alternative, the lung reduction device 120 may also be configured to be placed to advantage the user or practitioner in a manner that allows for subsequent removable through reverse and/or similar methods used for delivering and deploying the lung reduction device.

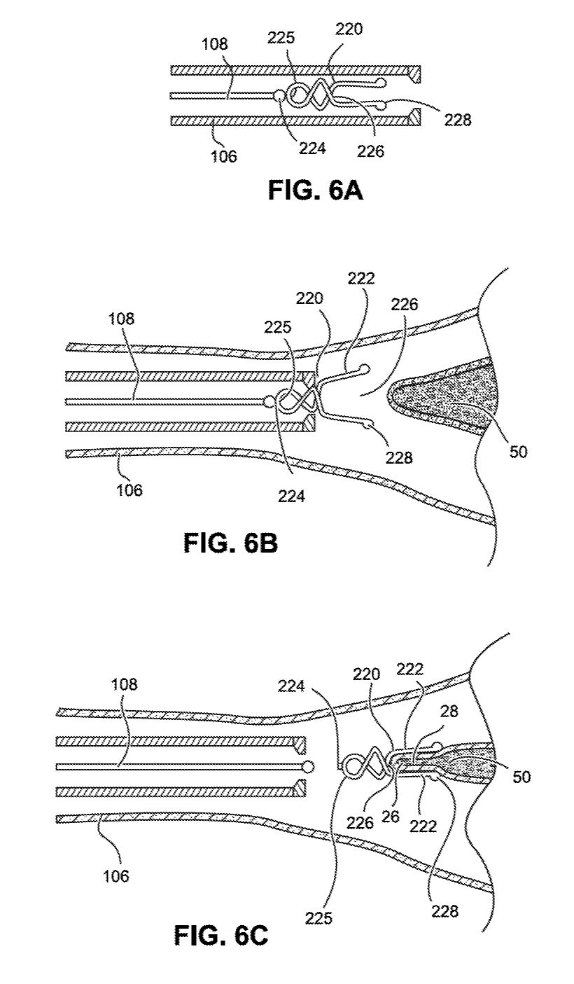

The lung reduction device shown in FIG. 4 may be dimensioned to straddle (in contact or slightly upstream of) selected forks or airway bifurcations 26. In at least one embodiment, it is envisioned that the bifurcating arms (or saddle 126) of the lung reduction device 120 may be configured to rest directly upon the corresponding forks 26 at the airway bifurcation. The saddle 126 may be dimensioned with a V-shape to maximize the force exerted between the arms 122. The saddle 126 may be dimensioned with a U-shape to maximize the depth to which the lung reduction device 120 is inserted distally into the airway 24 and reduce trauma to the airway bifurcation. A U-shape saddle 126 may also be preferred to maximize the contact surface area between the lung reduction device 120 and the walls of the corresponding airway 24. Additional shapes and configurations are envisioned to provide additional advantages such a coil spring configuration to increase the compression force of the device (See FIG. 6).

The deployment of the lung reduction device 120 follows the steps shown in FIGS. 5A-C. In FIG. 5A, the lung reduction device 120 is shown in the delivery configuration. The distal portion of the lung reduction device 120 consists of at least two arms 122 that are configured to be positioned in two or more branches 25 of the airway 24. The device is configured such that biasing force between the two arms 122 of the device is low enough to allow advancing of the device body by exerting a force upon the proximal nub 128 of the device body which results in the deployment of the device 120 into the small airways that define the septum 28. After target airway identification, the delivery tool 108 is brought into contact with the stem 124 (that can be a rounded proximal end) of the lung reduction device 120 for delivery through the airway 24 in proximity to the target area.

In some aspects, the delivery tool 108 (e.g. rod, pusher, grasper or gripper) may be configured to affect the tension between the arms 122 of the lung reduction device 120. Specifically, the delivery tool 108 may actuate a mechanism of the stem 124 or lung reduction device 120 to render the arms 122 inactive, or in a state of reduced tension. In such embodiments, the release of the stem 124 or lung reduction device 120 from the delivery tool 108 would actuate the arms 122 of the lung reduction device 120. In at least one aspect of the device of FIG. 5A-C, the tension is passively stored in the device body and arms 122 and a pusher or rod delivery tool 108 may be used to extend the device 120 from a catheter 106 and into place over the septum 28.

Once positioned, the arms 122 are able to exert the compressing force to reduce the volume of the diseased lung tissue 50 between the aims 122 of the lung reduction device 120. In further embodiments where a delivery tool 108 is more complex, such as a grasper or forceps that are used to deploy the device 120, it is envisioned that a rotation, ratchet action or other tool manipulation from the proximal end may be performed to release the stem 124. Once no longer attached or in contact with the delivery tool 108, the device may return to its natural deployed configuration.

The opening at the distal end of the catheter is positioned facing a bifurcation of the airway bifurcation 26 providing positioning of the lung reduction device 120 at the airway bifurcation. One or two diverging guide wires can be retained in the bifurcating airways to assist visualization on the fluoroscopic X-ray imaging device such as a C-Arm fluoroscope. In FIG. 5B, the lung reduction device 120 has been advanced, possibly with the use of a delivery tool 108, into contact with the tissue of the airway bifurcation 26. Upon further advancements, the arm 122 of the lung reduction device are spread apart and are able to advance and slide upon septum walls into the branches of the airway 24 holding and compressing the septum 28, or diseased tissue 50 at the bifurcation 26, in between.

The distal and proximal portions of the lung reduction device 120 are configured to remain in contact with the tissue of the airway 24 and therefore should be constructed to be atraumatic. Lung reduction device deployment is completed and the delivery tool 108 is removed from contact with the proximal stem 124 (proximal end) of the lung reduction device 120 (FIG. 5C).

In a further aspect, the deployment and implantation of a lung reduction device 220 may follow the steps shown in FIGS. 6A-C. In FIG. 6A, the lung reduction device 220 is shown in the delivery configuration. For the embodiment shown in FIGS. 6A-C, the biasing force between the arms 222 of the lung reduction device 220 may require leverage beyond simple tissue contact to overcome. As seen in FIG. 6B, the lung reduction device 220 comprises a device body between the vertex 225 and arms 222. The device is configured to interact with both a delivery tool 108 and the walls of the catheter 106 used in delivery. This interaction may temporarily widen the distance between the arms 222, as force is exerted upon the stem 224 of the device body. Finally, in FIG. 6C, as the catheter 106 is retracted away from the airway bifurcation, the lung reduction device 220 attempts to return to its original configuration, providing a strong biasing force to the walls of the airway bifurcation 26 held between the arms 222 of the device. This force pinches the tissue of the septum 28 of the airway bifurcation.

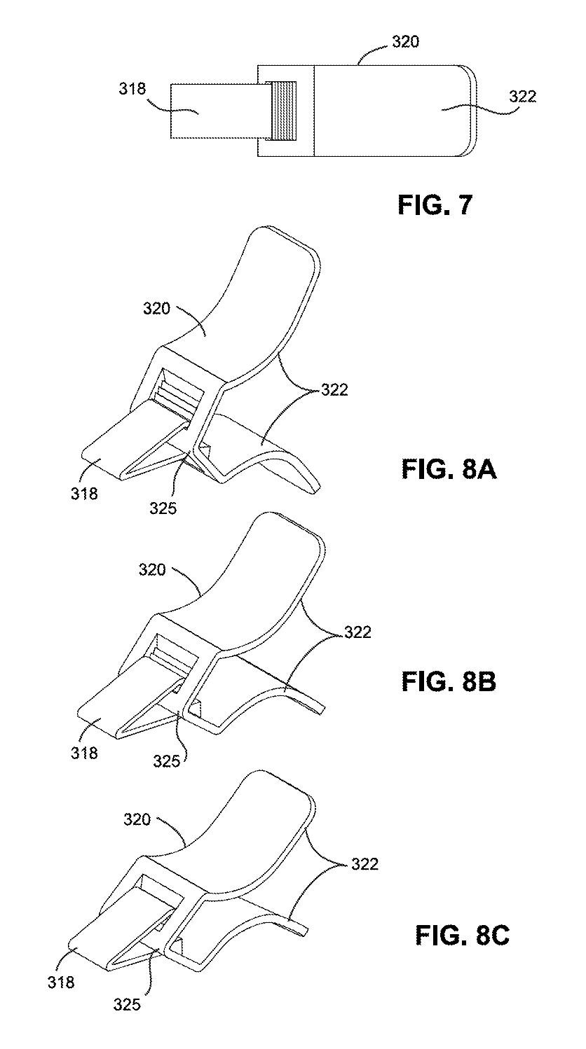

In a further aspect, a lung reduction device 320, as illustrated by FIG. 7 and FIG. 8A-C, may rely on an additional feature (e.g. a locking tab 318) to permanently increase the device biasing force once the device positioned. In FIGS. 7, 8A-C and 9A-C, a lung reduction device 320 with device body 321 and distally extended arms 322 is shown interacting with a second biasing locking tab 318. At rest or at a neutral position, the distal arms 312 of said device may be configured to split or diverge at various angles, splay freely or widen easily at the vertex 325 of the device body 321. In this variable (i.e. open) configuration, as the arms 322 are advanced over tissue into airway branches, the angle between said arms 322 would vary freely accommodate the tissue of the septum 28 (i.e. the tissue downstream of the bifurcation 26). Once the device is advanced sufficiently downstream over the septum 28 to capture an area of lung tissue, the device may then be activated or locked to switch from the variable to a static configuration. This switch may be actuated by a manipulation to the stem, saddle, or device body 321. FIG. 7 shows the top view of a locking tab 318 interacting with the first step of the proximal end of a lung reduction device 320.

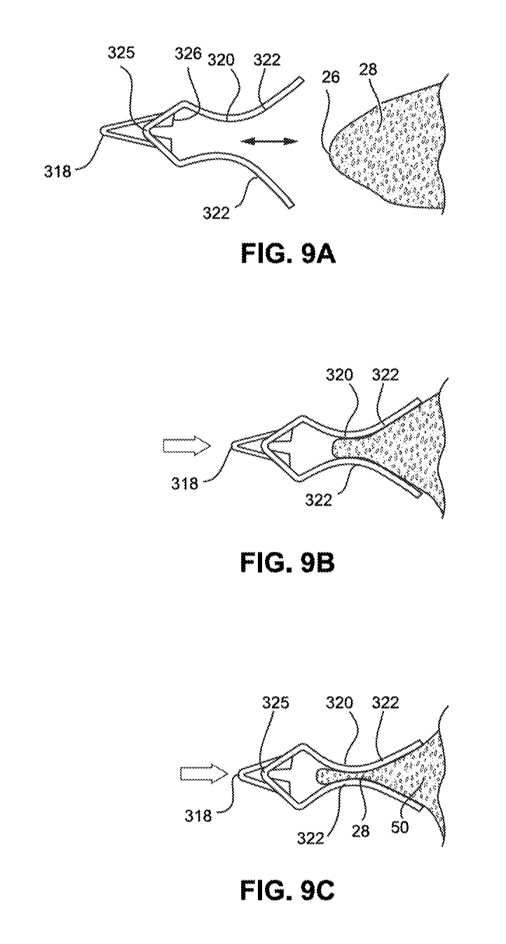

The deployment of the lung reduction device 320 with a locking tab 318 of FIG. 7 interacting with the device body 121 is shown in the sequential perspective views of FIGS. 8A-C along with the sequential elevation views of FIGS. 9A-C. FIGS. 8A and 9A show a lung reduction device 320 with arms in an open or variable configuration, prior to device deployment with the locking tab 318 contact, but not interacting with the device body 321. FIGS. 8B and 9B show a lung reduction device 320 with arms 322 in an intermediary stage or configuration, and with the locking tab 318 partially interacting with a locking mechanism, FIG. 9B also showing the partial reduction of the septum 28 volume. It is envisioned that the biasing force may be progressively increased as the locking tab 318 is driven toward the device body 321 of the lung reduction device 320.

Finally, FIGS. 8C and 9C show a lung reduction device 320 with arms 322 in closed configuration, with the locking tab 318 fully locked engaged with the locking mechanism, FIG. 9C also showing the ideal volume reduction of the lung tissue in this or a similar configuration. Notably the arms 322 of the device can be longer relative to the device width and the drawing in FIG. 9C and others are intended to illustrate the mechanism of action, not the mechanical dimensions of the device. In all the embodiments thus illustrated the arms may traverse several generation of airways.

As discussed, minimally invasive techniques are one envisioned method of device delivery and may include the use of a bronchoscope to help position and deploy the clip. A bronchoscope 104 is inserted through the mouth, trachea 10 and into the lung airways. A physician maneuvers a distal end of the bronchoscope and may be assisted by viewing an image of the lung airways 24 at the distal end captured with a miniature visual recorder that is held within the bronchoscope. Computed tomography (CT) and fluoroscopy imaging may also be used for imaging and navigation. An airway bifurcation 26 and septum 28 formed between two diverging lung airway may be identified and confirmed as a target for positioning the lung reduction device.

After identification of the selected airway bifurcation 26, the bronchoscope is navigated through branches of the lung airways 24 leading to the bifurcation 26. In addition to any visual recorder or camera which displays an image of the airway directly in front of the distal end of the scope, the tip may also deflect to assist the user in navigating the airways. The images can be used to maneuver the distal end through the trachea and larger sized braches 25 of the lung airways 24. As a result of large size relative to the catheter, the bronchoscope may not always extend to the desired smaller generations of airways with the target location and tissue 50.

To traverse the smaller braches, a catheter, guide wire, or combined system (not shown) is extended from the distal end of the bronchoscope 104 and maneuvered through the increasing smaller branches until the distal end of the delivery device extends to the selected bifurcation. A guide wire 102 may then be passed into a branch adjacent the selected bifurcation. The guide wire 102 can be used to navigate into distal airways 24 (too small for bronchoscope) under fluoroscopic guidance. When used, a catheter 106 travels along the path of guide wire 102 to maneuver the distal end of the catheter to the vicinity of the selected bifurcation. The guide wire 102 may extend through the distal tip portion of the catheter and be housed entirely within the length of the catheter. The guide wire 102 may be retracted after the distal end of the catheter is positioned near the selected bifurcation, or it may serve further use in positioning the device.

It is envisioned in some embodiments that no guide wire 102 is needed to position and deploy the device. In other embodiments, a single or dual-wire system may be used. Guide wire systems may have additional advantages in delivery, which are also described herein Similarly, when advantageous, a sheath or catheter may be used to deliver the device. As shown in FIG. 4, with the distal end of a catheter 106 housing the device and facing the selected bifurcation, the lung reduction device 120 (and optional delivery sheath, which is not shown) are advanced together out of the distal end of the catheter. A delivery tool 108 shown schematically on FIG. 5 may grasp, screw on or be otherwise releasable attached to the stem 124 of the lung reduction device 120. In at least one preferred aspect, the delivery tool specifically configured for this purpose may be configured to interface with the proximal spherical nub portion of the device. The grasping and release mechanism may in a shape that is advantageous to torque, turn or manipulate the distal end of the device.

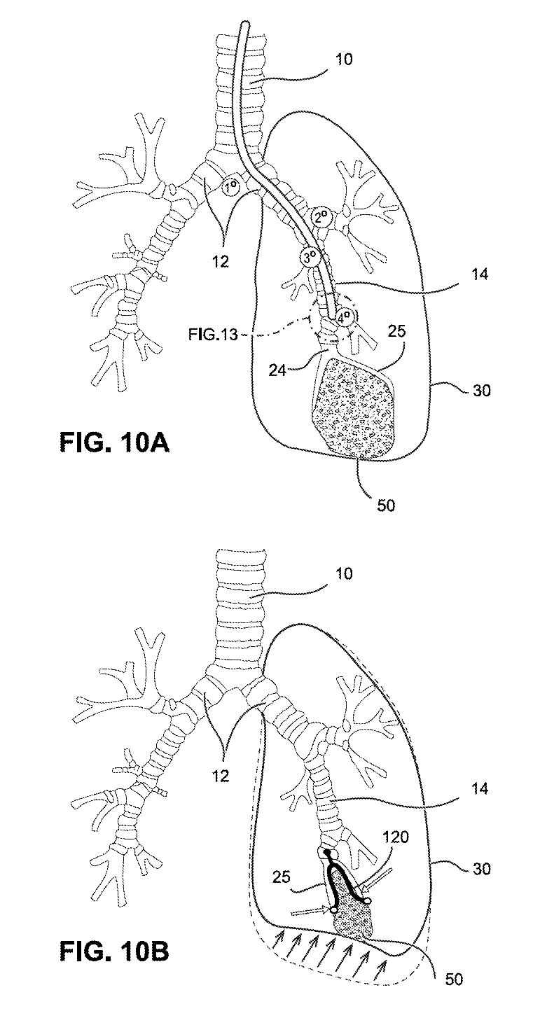

FIGS. 10A-B illustrates the before and after, showing the basic mechanism of the lung reduction process in which the lung reduction device(s) is implanted at a bifurcation 26 in the lung airways 24 and alters the anatomy of the lung airways 24. The lung reduction device 120 is advanced to said bifurcation 26, an airway bifurcation formed by the splitting of the airway 24 into two smaller, downstream branches 25. The arms 122 of the distal portion of the lung reduction device 120 are subsequently advanced into two or more branches of an airway 24 (FIG. 10A). It may be determined that the parenchyma of the lung between the branches of the airway 24 may be diseased parenchyma (i.e. diseased lung tissue 50). Diseased parenchyma can be characterized by a dramatic reduction in its elastic recoil capability and the trapping of air within the smallest airway and structures.

As the lung reduction device 120 is deployed (FIG. 10B), the lung reduction device 120 causes the branches of the airway 24 to move closer together, biased by compressing force exerted by the lung reduction device, and compress the target lung tissue. At the same time the surrounding non-targeted tissue is stretched and regains some of its elastic recoil. Once deployed, the delivery device (that can be forceps) is withdrawn from the lung airways 24. It can be repositioned to deliver another lung reduction device. Deployment of the lung reduction device results in bringing the branches of the airway 24 closer together and compressing the targeted diseased tissue 50 of the lung parenchyma to reduce total (overall) lung volume (FIG. 10B). The lung reduction device 120 is intended to compress targeted lung tissue 50, tension the surrounding diseased tissue, which increases elastic recoil and redirects air to healthier portions of the lung for more effective ventilation. The airways 24 are expected to remain patent or collapse later in exhalation, at a lower RV and result in more complete exhalation of air. This is expected to increase efficiency (e.g. metabolic efficiency) of the respiratory muscles (e.g. intercostal and diaphragm) and reduce the sensation of dyspnea at exercise that is debilitating to patients with air trapping and is associated with high RV. This therapy targets local diseased regions of the lung; therefore, one or more lung reduction devices may be necessary to achieve adequate effect.

While the measure of success of the lung reduction device described herein is determined most reliably on a case-by-case basis, benchmarks (measured or self-report data) such as lung volume, exercise tolerance and metabolic efficiency can be used to gauge the success across a wider patient population. For instance, in at least one instance, the invention may compose novel method of treating emphysema of the lung by reducing the RV of the air trapped in the lung by deploying a lung reduction device at the bifurcation of an airway 24 where the lung reduction device 120 is equipped with at least two arms that are inserted into the branching (i.e. bifurcating or trifurcating) airways and after deployment the lung reduction device exerts compressing force that compresses diseased lung tissue 50 and reduces volume of the lung at least partially restoring the elastic recoil. A measure of RV reduction may be one index to indicate the successful deployment of the device.

In another embodiment, the invention may comprise the novel method of improving exercise tolerance in the patients with emphysema by reducing RV of air trapped in the lung that consists of identifying patients with high RV, such as more than 50% to 250% above predicted value, lung reduction device in the bifurcation 26 of the airway 24 of the patient where the elastic lung reduction device at least partially resides in two bifurcating airways and exerts compressing force on diseased tissue 50 between the branching (e.g. bifurcating) airways 24 thus reducing the lung volume.

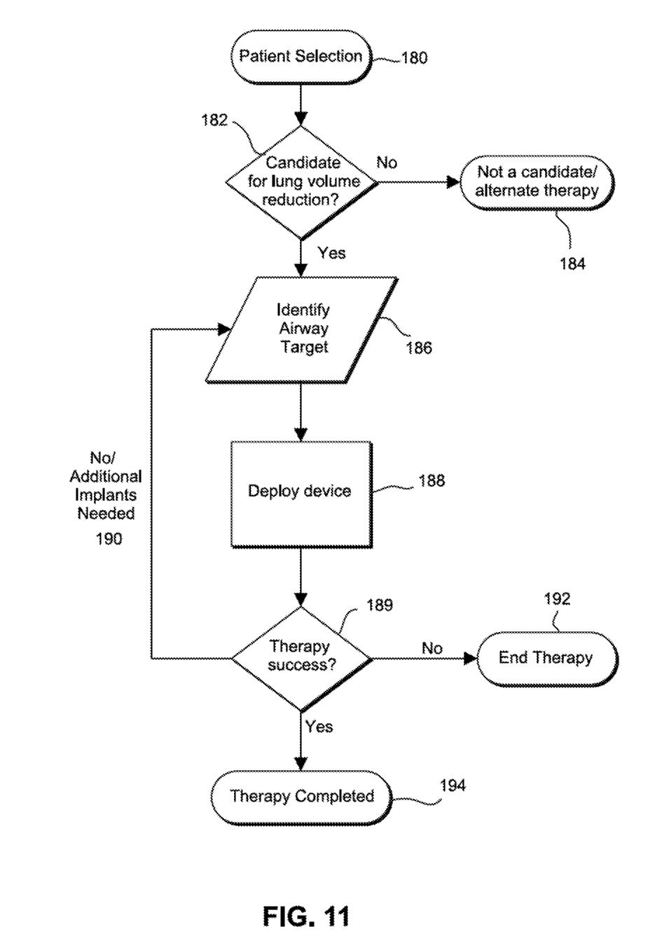

FIG. 11 shows a flowchart illustrating a method of treating the lung according to the embodiments of the invention. The initial step in treating the lung includes patient selection 150. Patients having a diagnosis of severe emphysema with disabling dyspnea, moderate-to-severe obstructive defect, high RV such as 100 or 200% of predicted value, and limited exercise capacity that can be indicated by reduced distance walked in six minutes (i.e. Six-Minute Walk Test) are considered good candidates. Evaluation of lung function may be performed prior to treatment to select appropriate candidates for therapy. Lung function may be evaluated by using a ventilator attached to a patient, using one or more imaging modality such as CT, using a blood oxygen sensor, and/or using a treadmill or other exercise stress testing. Some of these evaluation methods, particularly an oxygen sensor (pulse oximeter), ventilator and/or imaging systems may be used to both evaluate pre-treatment lung function as well as monitor one or more parameter during the procedure. A ventilator can provide information regarding lung function, such as pressure, volume, and/or flow.

In some embodiments, the thoracic cavity, including the lungs and diaphragm, can be imaged to evaluate and/or verify the desired lung characteristics, which may also comprise a shape, curvature, position and orientation of the diaphragm, and localized density or a density distribution map (visualize diseased portions of the lung). The thoracic cavity may be imaged using fluoroscopy, X-rays, CT scanners, PET scanners, and MRI scanners or other imaging devices and modalities. Pretreatment image data may be processed to provide a comparison to those measurements taken during and after the procedure. Patients with radiologic evidence of lung hyperinflation with flat diaphragms on chest X-Rays and areas of severe emphysema intermingled with better preserved lung tissue (heterogeneous emphysema) on CT scans are candidates for lung volume reduction 182. Patients not deemed a candidate may be offered alternative therapeutic interventions 184.

Upon identifying a candidate for lung volume reduction through patient selection 180, a targeted portion of the lung is treated by identifying the airway 186 in close proximity to the targeted portion of the lung. The lung reduction device is deployed 188 compressing one or more portions of disease tissue 50 to provide the desired therapeutic effect. Evaluation of the lung function after the targeted portion of the lung has been compressed is performed to determine the efficacy of the treatment throughout the procedure. A determination of therapy success 189 is made based on the total lung volume reduction and a decrease in symptoms, which may be self-reported in some instances. Additional implants may be delivered and deployed 190 as described above until desired lung function or therapy success 189 is achieved. Upon achievement of the desired lung function, the therapy is concluded 194. The procedure may be aborted 192 or the lung reduction devices may be removed if the therapy is determined to be unsuccessful.

As illustrated by FIG. 12A-D in one envisioned embodiment, a clip device 420 may further comprise one or more a locking mechanisms (ridge 418 and collar 416 mechanisms both shown) positioned on or surrounding one or more of the arms. To reduce the device profile for delivery, it is envisioned that the device arms 422 may be temporarily compacted, as shown in FIG. 12A. It is envisioned that the device 420 may be positioned for delivery with arms open, as shown in FIG. 12B. When used, the collar 416 could revert to a proximal position along the device body to allow each aim 422 expand and enter a distinct airway during device delivery. Once the device is positioned with one arm 422 in each airway 24 surrounding the tissue held in between, the locking mechanism 118 of this embodiment would be actuated by a twisting motion used to rotate the vertex 425 of the device. A rotation of substantially 360 degrees (330-390 degrees), to the vertex 425 of the device 420 would cause the notched locking mechanism 118 of FIG. 12A-D to engage the opposite arm, as shown in FIG. 12B in an intermediate or transition step. This rotation of the device body may be performed by a delivery tool 108 (e.g. graspers, claspers, pusher, etc.)

FIGS. 12B and 12D show the first (opened) and second (locked) orientations of the device. It is envisioned that the number of partial or full rotations of the vertex required to actuate and release the locking mechanism may vary as a result of the dimension of the clip and the configuration of the device body 421 and arms. Based on the position and dimensioned of the locking mechanism 118, the exact degree of rotation may vary slightly, but the continued rotation of the device past the final locked position will cause the device to return to the open position of FIG. 12B. It is envisioned that an external mechanism, such as a collar 416 may be used to gradually increase or maintain the compressive force of the arms for device delivery, but may also help secure the arms 422 with sufficient compressive force if positioned distally after device deployment. Once placed, the collar 416 may remain in place in an extended or permanent manner to, as needed to maintain the compression achieved during device delivery. It should be understood that twisting of the vertex of the device body 421 (shown completed in FIG. 12D) not only transition the device body 421 and arms 422 into a locked orientation, but may actuate the locking mechanism while increasing the compressive force exerted by the arms on the tissue held in between. While the locking mechanisms are illustrated in combination, it is understood that each may be used independently or in combination with other aspects of the device.

In certain aspects, the dimensions of the device, including the device body 421 and arms may vary, as needed, to conform to variations in airway dimensions. The dimensions defined by illustration in FIGS. 12A-C represent one envisioned embodiment. Generally, the total length, lₜₒₜₐₗ, includes both the maximum arm length and the length of the device body, l₃. The arm lengths, l₁ and l₂ may vary, but may be comparable in some instances to maximize the compressive force exerted by the arms. The width of the device can be determined at certain lengths of the device, with height of the device at the proximal end shown as d₁. The distance between the device arms immediately distal the saddle is defined as d₂. In an embodiment where the device have a first and second orientation, it is envisioned that d₂ would be substantially reduced when the device is configured in the second or locked orientation. Finally, the distance between the device arms, measured at the terminus of the shorter or both arms 422, can be defined as d₃ and it is envisioned that the distance would generally be reduced as the device is configured in the second or locked orientation to increase the compressive force exerted by the arms.

The relatively ease of transition between the stages shown in FIGS. 12A-C may provide for several notably advantages, a number of which are described. A built-in locking and unlocking mechanism 418 would allow for the device to be delivered in a compressed form, if needed to navigate the later generations deep within the lung. The locking mechanism is embedded within the device and the device does not utilize a secondary locking mechanism, reducing the complexity of the delivery, operation and maintenance of the device. The reversible actuating steps allow the device to be position, re-positioned and removed, if necessary, without added complexity. Further advantages of the device and its associated methods of use may become apparent during the application and use of the device.

Another challenge that faces device delivery are the current dimensions needed to accommodate minimally invasive devices and techniques. Generally, a bronchoscope 104 equipped with visualization (e.g. camera 105) and a large working channel 2-3 mm in diameter cannot proceed distally or downstream beyond the 4^{th} generation of airways. A bronchoscope 104 without a large working channel 110 can pass several generations deeper into the lung, but does not have the capabilities of larger scopes Similarly, catheters, sleeves and guide wires can each penetrate deeper into the lung, but face various limitations in each case. In some aspects, it is envisioned that the delivery of the clip may be performed with or without the use of one or more guide wires. However, when used, guide wires or guide wires used as a part of larger delivery system, may be able to overcome the size limitation faced with bronchoscope delivery, and may simultaneously produce several unexpected advantageous.

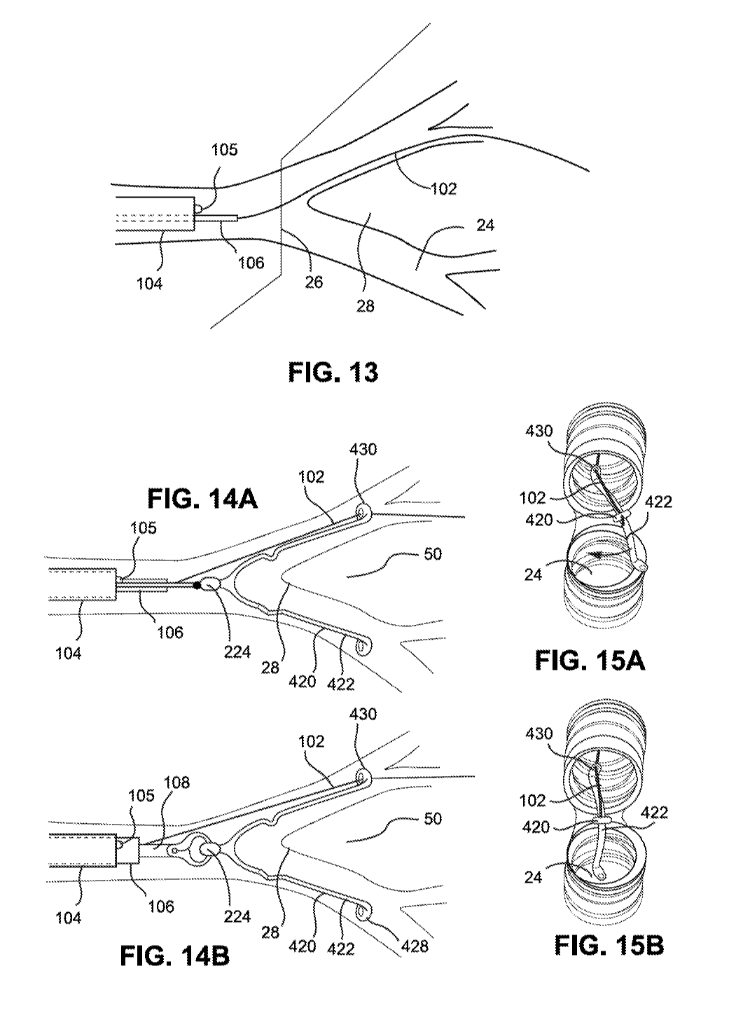

In at least one embodiment, a single guide wire 102, like the one shown in FIG. 13, may be extended through the working channel 110 of a bronchoscope 104 and beyond the dimensional limits of said bronchoscope. The wire 102 may be advanced into an airway 24 beyond the septum 28 past the target location and may subsequently be used to deploy a clip device. It is envisioned that a clip may comprise a device body 421, and two arms, and be configured to accept a guide wire 102 along a combination of channels within the arms or device body 421 to facilitate delivery along the guide wire 102. In at least one embodiment, the guide wire 102 would be configured to remain threaded in a distal arm eyelet, which is located at the distal end of the arm 422 of the lung reduction (i.e. clip) device 120 (See FIG. 4). Additionally, in other aspects, the device body 421 may contain a proximal body eyelet at the proximal end of the clip, dimensioned to receive the diameter of the guide wire (See FIGS. 14 A and B). It is envisioned that the proximal eyelet may alternatively be a groove to accommodate the guide wire 102. While the device is maintained along the length of the guide wire, the clip is able to traverse the airways 24 following the track laid by the guide wire 102 in a manner similar to a monorail or railroad train following a track.

FIG. 14A shows a clip device with a guide wire 102 passing through the arm eyelets of the device. The device has travelled a length of the guide wire 102 and has come to rest, positioned approximately at the delivery location and surrounding tissue 50. Specifically, each arm 122 of the device is positioned in a distinct airway 24 and surrounds the septum of a bifurcation 26 or airway bifurcation. While in FIG. 14A, a pusher is shown as the delivery tool 108 used to advance the clip in the distal direction and towards the septum 28, alternative delivery tools 108 including graspers (claspers, clamps, etc.) and various keyed devices may be used, especially if the proximal end of the device is configured to be rotated to align with the septum. An alternate delivery tool 108, such as a grasper, mounted on the distal end of the tool 108 activated by the operator from the proximal end of the system is illustrated by FIG. 14B. This rotation, which can be made around the axis defined by the guide wire illustrated in FIG. 14B, may be used for several functions, including aligning the arms with the airways before fully advancing the clip, reorienting a (e.g. vertex 125 actuated) device from a first to a second orientation, or rotating the clip, as needed, as described in the positioning of some delivery methods. In some embodiments, it is envisioned that the reorienting of the vertex of a device may occur in a step-wise manner similar to that shown in FIGS. 12A-C.

FIGS. 15A-B show an overhead cross-sectional view looking distally into the airways at a bifurcation. In FIG. 15A, the clip has not been positioned to be advanced into each bifurcating airway. A rotation of the device, whether by a delivery tool 108 or by another mechanism, results in an orientation as shown in FIG. 15B. The proper orientation will allow each arm 422 to be advanced into a separate airway 24.

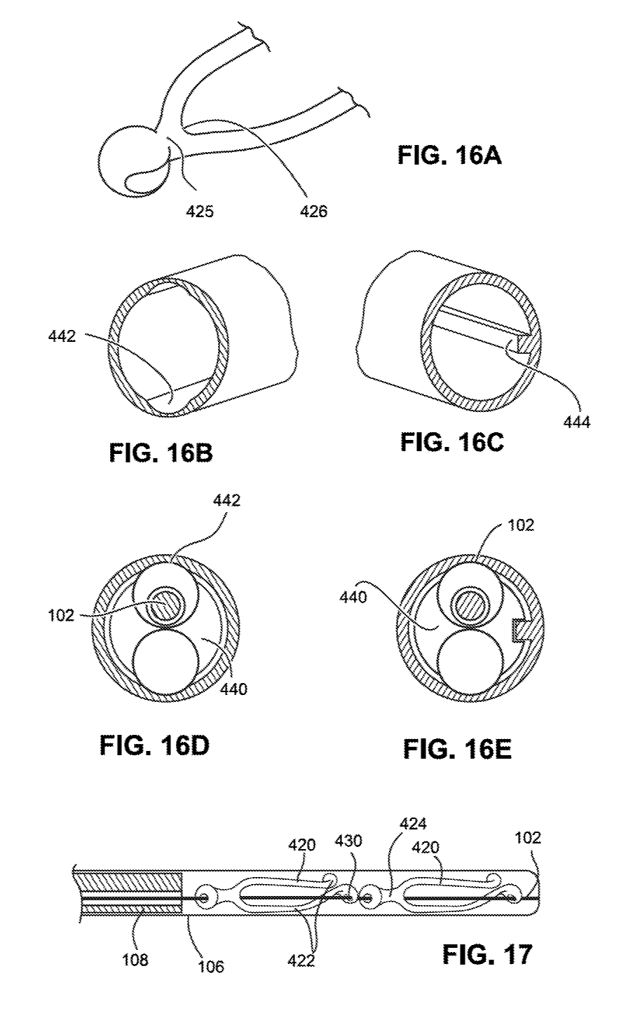

In an envisioned embodiment, a lung reduction device (i.e. clip) may be configured, as illustrated in FIG. 16A to slide along the grooves in the inner surface of the surrounding catheter, shown in FIG. 16B-C. The inner channel of the catheter may be rifled, notched, grooved, or dimensioned to allow an adapted device to be delivered along a single pre-determined pathway. Furthermore, the clip may be equipped with a key to rotate it from the proximal end to align it in the airway by rotating the catheter inside the working channel 110 of the bronchoscope 104, thereby aligning the clip. A combination of multiple pathways can ensure device delivery along a pre-determined pathway. FIG. 16D compares a grooved pathway to the grooved and notch combination of FIG. 16E, which requires an additional key to actuate the rotation of the device 420 for device delivery. The envisioned clip would be dimensioned to accept a key or other specifically shaped delivery tool to rotate or align it in the airway with each arm positioned to be advanced into a separate airway. A delivery sheath or catheter containing the clip could be torqued or rotated to achieve the proper alignment. Finally, the key may also be used to rotate the vertex to actuate the locking mechanism of a device.

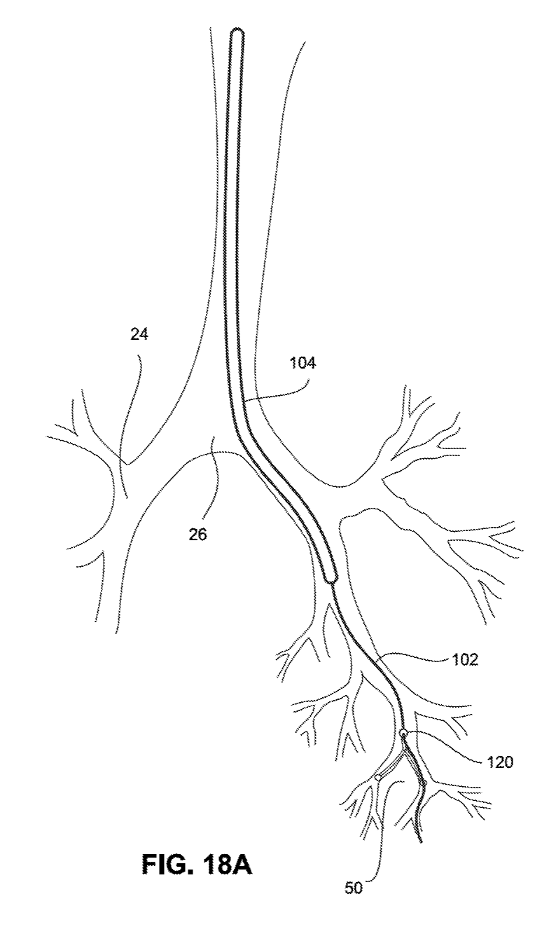

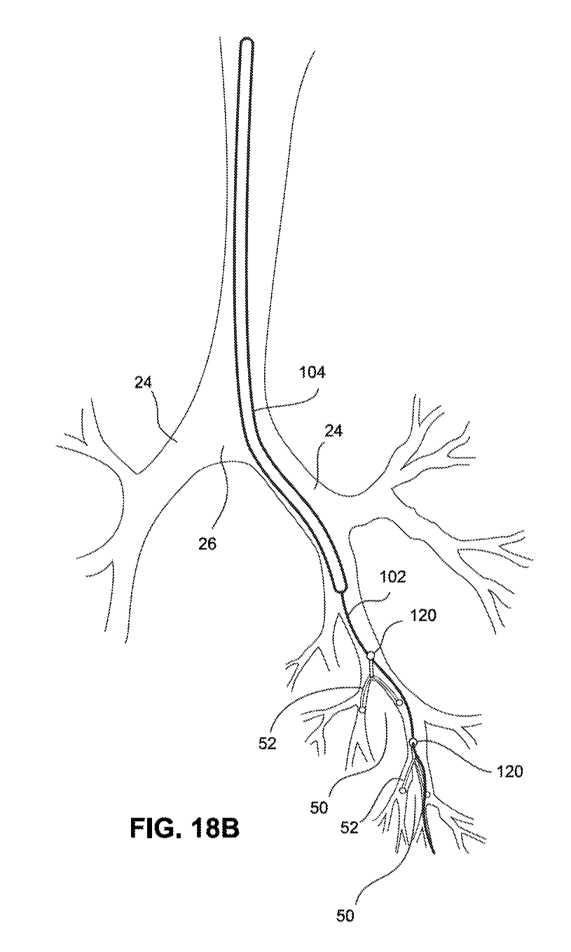

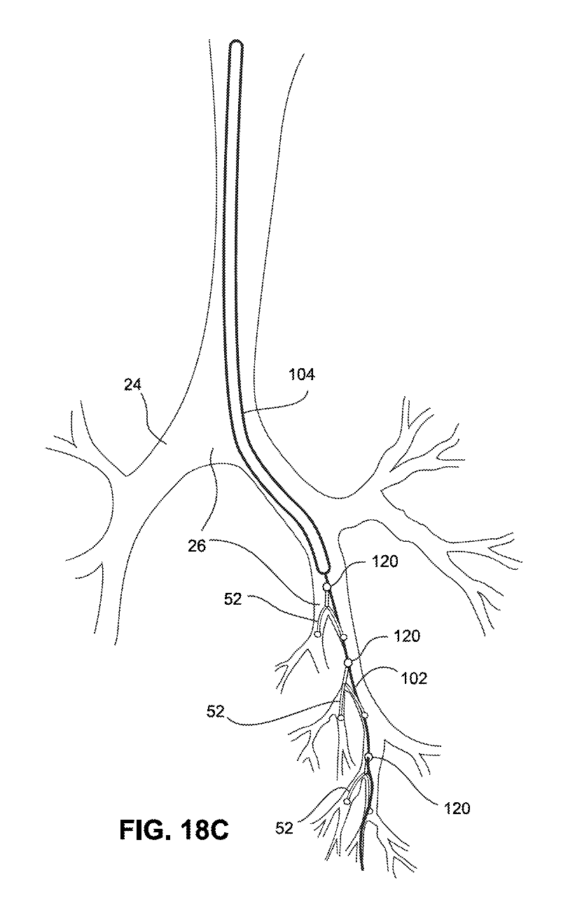

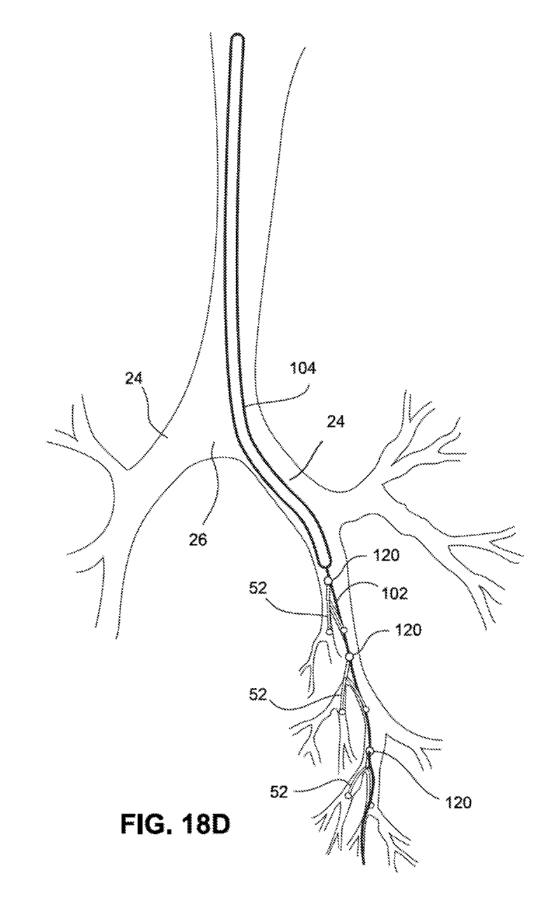

A grooved, over-wire delivery system may also assist in providing rapid sequential delivery of clips to target areas. For example, a clip device may be configured with elements (e.g. groove, notch or specific shapes, etc.) to help guide clip during the delivery of a first clip while simultaneously positing a second clip to take the position of the first clip upon delivery without need to withdraw and reposition the guidewire. FIG. 17 shows two clips held sequentially in a catheter for rapid delivery. More clips can be thus preloaded into the delivery system. Importantly the guide wire 102 is advanced deep into the bronchial tree passing several generations of bifurcations suitable to be “clipped”. The wire that can be as thin as commonly used (e.g. 0.0005 inch, 0.001 inch or 0.0032 inch wires) and may be equipped with a tapering soft or atraumatic tip that may be used to reach towards the very outer edge of the lung or pleura. The delivery tool 108, a pusher in this case, would be used to advance the front-most clip, while simultaneously loading the subsequent device 420 into a distal position for the next clip delivery. Such a delivery would be advantageous to decrease the amount of time required for the placement and application of the devices. An overall reduction of time needed for a surgical procedure generally reduces the risk to the patient and the overall cost. It is further envisioned that full sections of lobes or full lobes could be compressed over a short period of time using sequential delivery from a single guide wire. The frame-by-frame sequence of FIGS. 18A-D show the volume reduction that is envisioned from the use of multiple clips to compact tissue between bifurcating airways along a single guide wire. The clips shown in FIGS. 18A-D may be delivered and subsequently deployed before providing the biasing force needed to collapse the airway. It is envisioned that this approach could also be used along the multiple airways spanning a single lobe. Generally, the treatment of several lobes requires the procedures to be staged to allow the patient ample recovery time between surgeries.

In FIG. 18A a single clip is positioned with guidance from a single wire that has been advanced distally from the working channel 110 of a bronchoscope 104 and into an airway. The first clip is positioned with each arm 422 in a separate airway and the saddle advanced towards and over the septum of the bifurcation. Subsequently, as shown in FIG. 18B, once the first clip is positioned and deployed to compress and at least partially collapse the diseased tissue 50 captured between each arm, a second clip may be advanced distally towards the next or a further upstream bifurcation.

In FIG. 18C, three clips have been advanced into the airway along the path established by the guide wire. The two distal-most clips have been advanced and deployed, and the volume of diseased lung tissue 50 held within the arms of the clips has been reduced through the bias force provided by the clip. Finally, FIG. 18D shows three clips advanced and deployed within the airway along the path established by the guide wire. It is envisioned that the process of positioning and deploying multiple airway clips could be repeated, as needed, to maximize the volume reduction during a single procedure. In some instances, deploying multiple clips within the airways of one lobe may be sufficient to reduce the volume of the lobe and improve or restore function to the remaining, relatively healthy lobes. In other instances, the use of multiple clips in additional lobes may be required to achieve the minimum (10-15%) volume reduction needed before an improvement in lung dynamics is manifested.

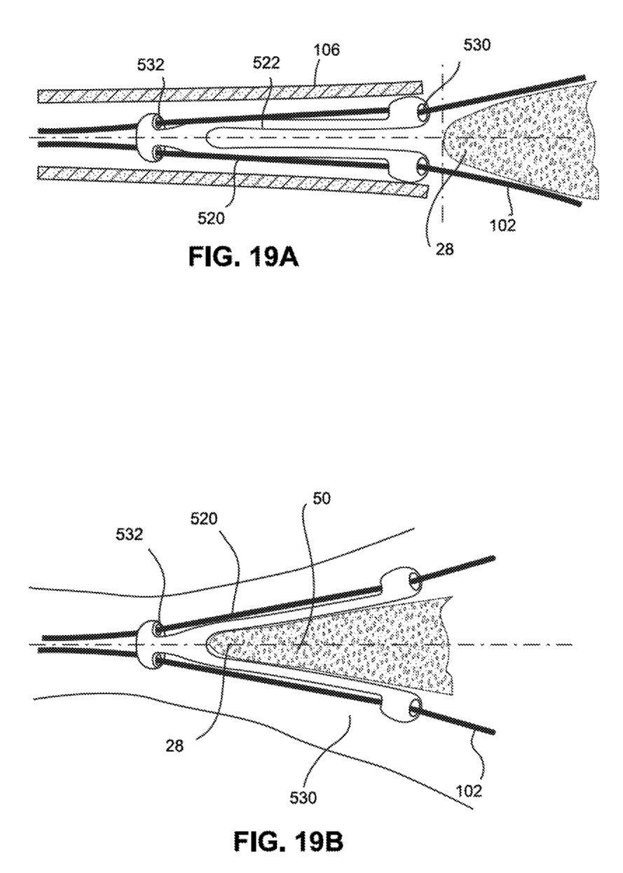

While the benefits of single guide wire assisted delivery have been described, it is envisioned in at least one alternative embodiment that dual guide wires may be used to position and deploy a specifically configured airway clip. Such a clip, as shown in FIGS. 19A and 19B, may have eyelets 532 at the distal end of each arm 522. In addition, some embodiments may also pass each guide wire 102 through an opening, or a groove configured to accommodate the wire, at the proximal end of the device body 521. In an alternative, the device body 521 could comprise a single eyelet 532 or a channel to accept both wires. Certain advantages may be made possible with the use of two guide wires 102 and include the ability to automatically align the clip during deployment without the need for precise positioning. This advantage would also allow for deployments to occur faster and with greater force against the septum, which may optimize the distal positioning of the device. In addition, a second wire 102 might assist in positing the device within separate airways 24 without the need for adjustment or corrective rotation. Dual guide wire methods 102 are also envisioned to work in combination with many of the other aspects described herein.

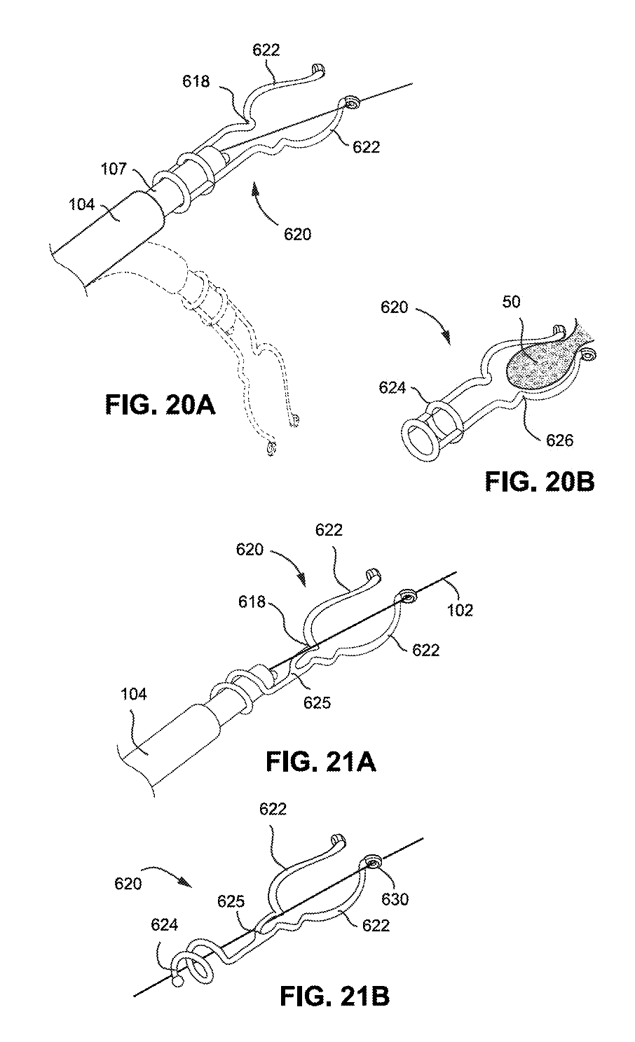

In some instances, it may be advantageous to further reduce the dimensional requirements needed to deliver one or more clips to a target location. By reducing or eliminating the working channel 110, the diameter of a bronchoscope 104 may be greatly reduced while maintaining visualization and navigation ability. Thus, the smaller bronchoscope 104 can advance deeper into the lung. FIG. 20A shows one or more clips loaded onto the exterior of a bronchoscope 104 to enable the direct delivery of the lung volume reduction mechanism to deeper lung areas. One aspect of the device is depicted with a minimalized working channel 110, dimensioned to accommodate only a guide wire 102, which may assist device delivery when present. This device 620 and guide wire 102 may be designed to enable the wire to “thread” through the eyelet 632 in the arm 622 of the clip when it exits the bronchoscope. Such a reduction of the bronchoscope 104 diameter would allow a bronchoscope (equipped with visualization) to be advanced distally several generation deeper than a bronchoscope 104 with both a large working channel 110 and camera 105. As the scope is still equipped with visualization, a deflectable bronchoscope 104 may be used to position the device with or without the use of a guide wire.

It is further envisioned that the device body 621 of the device 620 could be configured to allow the clip device to be grasped by a delivery tool 108 to assist in device positioning, deployment or removal. In is envisioned that with a reduced or entirely absent a working channel, the device delivery could be performed by loading the clip or clips to the exterior of the scope, as shown in FIG. 21A-B. While a delivery tool 108 may still be used, in some embodiments of the device 620 using over-scope delivery, it is envisioned that the device body 621 of the clip may be configured to be delivered from the distal tip of the bronchoscope as the result of a simple pushing mechanic Reorientation of the device to a second locked orientation may be performed by a delivery tool 108, the delivery catheter 106, or automatically during delivery as the placement of the device. Once again the distal tip of the bronchoscope may be articulated (not shown) to finely adjust the positioning of the clips. Delivery may be performed directly by the scope under direct visualization before the clip or clips are slid into place.

In yet another aspect, as seen proximal to the distal region of the device body 621 shown in FIG. 21A-B, a catheter 106 or sheath assisted delivery is also envisioned to be able to assist in device 620 positioning to assist sliding the clip off the shaft of the bronchoscope 104. The device 620 and body 621 may further form a spring that interfaces tightly with the bronchoscope 104 shaft so as not to be dislodged unintentionally. A spring release mechanism can be engaged to facilitate sliding of the device 620 from the shaft. While a bronchoscope approach is generally considered to be safe and effective, the dimensional limitations present as a result of working within the airways of the lung may favor the use of smaller catheters or delivery sheaths.

In at least one aspect, it is envisioned that a delivery sheath may be used in combination with an over-scope delivery to house the clip until it has been correctly positioned. In another aspect, a catheter 106 may be advanced alongside a bronchoscope 104 to provide a second working channel for purposes including wire or device delivery.

While at least one exemplary embodiment of the present invention(s) is disclosed herein, it should be understood that modifications, substitutions and alternatives may be apparent to one of ordinary skill in the art and can be made without departing from the scope of this disclosure. Each embodiments described and illustrated in the figures may be shown with repeat-reference numerals, with the understanding that each embodiment can be viewed independent from each other. This disclosure is intended to cover any adaptations or variations of the exemplary embodiment(s). In addition, in this disclosure, the terms “comprise” or “comprising” do not exclude other elements or steps, the terms “a” or “one” do not exclude a plural number, and the term “or” means either or both. Furthermore, characteristics or steps that have been described may also be used in combination with other characteristics or steps and in any order unless the disclosure or context suggests otherwise. This disclosure hereby incorporates by reference the complete disclosure of any patent or application from which it claims benefit or priority.

1.-53. (canceled)

54. A method of reducing the volume of the lung tissue associated with a first and second airways of an airway bifurcation in a patient, the method comprising:
loading an implantable airway device with at least one alternate orientation to the exterior of a bronchoscope,
advancing the device-loaded bronchoscope into the airway until proximal to the airway bifurcation in the lung,
further advancing the device to a distal position with a first arm of the device in a first airway and a second arm of the device in second airway, and
manipulating the device while the first arm is in the first airway and the second arm is in the second airway, wherein the manipulation of the device orientation results in a bias applied by the first and second arms to the first and second airways sufficient to reduce the volume of the lung associated with the first and second airways.

55. The method of claim 54 further comprising retracting the bronchoscope from the patient airway without the device being attached to the bronchoscope.

56. The method of claim 54, wherein the implantable airway device includes a vertex, the first arm having an end connected to the vertex and the second arm having an end connected to vertex, wherein the further advancing of the device includes positioning the vertex adjacent the airway bifurcation.

57. The method of claim 56 wherein the manipulation of the device includes turning the vertex.

58. The method of claim 54, wherein the further advancing of the device includes advancing the device from a sheath attached to the bronco scope.

59. The method of claim 54, further comprising advancing a guide wire from the bronchoscope, through the airway, past the airway bifurcation and into the first airway, and thereafter the further advancing of the device includes sliding the device arm along the guide wire as the first arm advances into the first airway.

60. The method of claim 59, wherein the implantable airway device is a first device and the method further comprises, after the manipulation of the first device, advancing a second implantable airway device from the bronchoscope along the guide wire such that a first arm of the second implantable airway device is positioned in one of the airways.

61. The method of claim 60, further comprising advancing a vertex of the second implantable airway device to position adjacent a second airway bifurcation of the airways.

62. A method loading an implantable airway device into a sheath of a bronchoscope, the method comprising:
positioning an implantable airway device in the sheath, wherein a vertex of the device is oriented proximally in the sheath and arms of the device are oriented distally in the sheath, such that the arms of the device face towards a distal open end of the sheath, and
positioning a delivery tool in the sheath such that the delivery tool is proximal in the sheath with respect to the implantable airway device, wherein the delivery tool is configured to move the implantable airway device relative to the sheath to deploy the implantable airway device into an airway of a mammalian patient.

63. The method of claim 62 wherein the implantable airway device is a first device and the method further comprises positioning a second implantable airway device in the sheath between the delivery tool and the first device.

64. The method of claim 62, further comprising installing a guide wire in the sheath such that the guide wire extends through an opening in at least one of the implantable airway devices.
